(19) United States
(12) Patent Application Publication
(10) Pub. No.: US 2023/0407339 A1
(43) Pub. Date: Dec. 21, 2023

(54) TRANSFERABLE TYPE I-F CRISPR-CAS GENOME EDITING SYSTEM

(71) Applicant: The University of Hong Kong, Hong Kong (CN)

(72) Inventors: Aixin Yan, Hong Kong (CN); Zeling Xu, Hong Kong (CN)

(21) Appl. No.: 18/251,374

(22) PCT Filed: Nov. 4, 2021
(86) PCT No.: PCT/CN2021/128715

§ 371 Date: May 1, 2023

Related U.S. Application Data
(60) Provisional application No. 63/110,683, filed on Nov. 6, 2020.

(51) Int. Cl. C12N 15/90; C12N 15/11; C12N 9/22; C12N 9/12; C12N 15/78; C12N 1/20

(52) U.S. Cl. CPC C12N 15/902; C12N 15/11; C12N 9/22; C12N 9/1241; C12N 15/78; C12N 1/20; C12N 2310/20; C12N 2800/80; C12N 2800/101

(57) ABSTRACT

A generic type I CRISPR-Cas-based genome-editing system that can be used in microbial hosts having diverse genetic backgrounds has been established. The chromosomal integration system overcomes the limitations of narrow host range and the requirement for antibiotics to maintain propagation and expression which are associated with plasmid-encoded Cas proteins. Compositions and methods for a chromosomal integrated type I-F CRISPR-Cas system for programmable genome editing and robust gene regulation are provided. In some embodiments, the compositions and methods are effective to selectively and specifically edit and/or regulate the genome of multiple microbial species with diverse genotypes. Compositions and methods for gene editing and/or gene regulation in Pseudomonas spp, such as multiple strains of P. aeruginosa, are described.

### FIELD OF THE INVENTION

The invention is generally directed to the genetic modification of microorganisms, specifically using a transferable CRISPR-CAS-based genome editing system.

### BACKGROUND OF THE INVENTION

Clustered Regularly Interspaced Short Palindromic Repeats (CRISPR), and associated CRISPR proteins (Cas) provide bacteria with adaptive immunity against foreign genetic elements, such as bacteriophage infection (Marraffini, et al. Nature 526, 55-61, doi:10.1038/nature15386 (2015)). Dependent on a small CRISPR RNA (crRNA) to achieve site-specific DNA targeting and interference, the application of CRISPR-Cas-mediated genetic engineering systems has revolutionized the field of genetics with its great abilities of specificity and re-programmability (Adli, Nature Communications 9, doi:10.1038/s41467-018-04252-2 (2018)).

CRISPR-Cas systems are typically classified into two classes, based on the different compositions of their effector complex (Makarova, et al. Nature Reviews Microbiology 18, 67-83, doi:10.1038/s41579-019-0299-x (2020)). Type I systems employ a multi-subunit effector complex which is known as the CRISPR-associated complex for antiviral defense (Cascade) to interfere with DNA or RNA, and Type II systems are distinguished by a single effector with multiple domains (Koonin, E. V. & Makarova, K. S. Philosophical Transactions of the Royal Society B-Biological Sciences 374, doi:10.1098/rstb.2018.0087 (2019)).

Specifically, the single-effector class 2 systems, such as Cas9 and Cas12a, are widely applied for various basic and clinical applications including genome editing and diagnostics in a broad range of eukaryotic organisms (Knott & Doudna, Science 361, 866-869, doi:10.1126/science.aat5011 (2018); Zhang, F. Quarterly Reviews of Biophysics 52, doi:10.1017/s0033583519000052 (2019)). However, although single-effector type II CRISPR-Cas9-mediated systems have been successfully applied for genetic manipulation of specific model prokaryotes, the application of these systems in non-model species/strains, including the majority of clinically or environmentally isolated strains, is problematic. This is largely due to the high level of diversity in DNA homeostasis amongst microbial cells, together with the cytotoxicity of over-expressing heterologous Cas9 proteins in certain genotypes.

Type I systems are the most abundant (˜90%) CRISPR-Cas systems presented in both bacteria and archaea, and represent a great potential for diverse and flexible applications by their versatile properties. For instance, multiple subunits in the Cascade complex can be fused with transcriptional modulators without functional disruption. Pre-assembled T. fusca type I Cascade ribonucleoprotein (RNP) and Cas3 can be delivered into human cells to achieve long-scale genomic deletions, and gene editing in multiple human cell lines with high specificity and efficiency using a two-component expression vectors encoding the type I Cas proteins and the guide crRNA, respectively, has been demonstrated (Pickar-Oliver, A. et al., Nature Biotechnology 37, 1493-1501, doi:10.1038/s41587-019-0235-7 (2019); Chen, Y. et al., Nature Communications 11, 3136, doi:10.1038/s41467-020-16880-8 (2020); Dolan, A. E. et al. Molecular Cell 74, 936, doi:10.1016/j.molcel.2019.03.014 (2019); Cameron, P. et al. Nature Biotechnology 37, 1471, doi:10.1038/s41587-019-0310-0 (2019)). Nevertheless, to date only a few type I systems have been utilized for genome editing in microorganisms. Type I-A, I-B, I-E and I-F systems have been reported in S. islandicus, C. pasteurianum, L. crispatus, and P. aeruginosa, respectively (Li, et al. Nucleic Acids Research 44, doi:10.1093/nar/gkv1044 (2016); Pyne, et al., Scientific Reports 6, doi:10.1038/srep25666 (2016); Hidalgo-Cantabrana, et al., Proceedings of the National Academy of Sciences of the United States of America 116, 15774-15783, doi:10.1073/pnas.1905421116 (2019); Xu, et al. Cell Reports 29, 1707-, doi:10.1016/j.celrep.2019.10.006 (2019)). A native type I-F CRISPR-Cas system encoded in a clinically-isolated, multi-drug resistant P. aeruginosa strain (PA154197) has been shown to be reprogrammable for high-efficiency in situ genome editing. However, these applications are limited to specific microbial hosts containing an active and extensively studied endogenous CRISPR-Cas system. Extending these applications to other strains even belonging to the same species is highly error-prone, and these methods are not applicable to the majority of strains of the highest clinical, industrial, or environmental importance which do not contain any CRISPR-Cas system (˜50% of strains), or which contain a degenerated, non-functional CRISPR-Cas system (˜40% of strains) (Selle & Barrangou, Trends in Microbiology 23, 225-232, doi:10.1016/j.tim.2015.01.008 (2015)). Even those strains that can be potentially engineered using plasmid-encoded Cas proteins, it suffers from several limitations, such as the requirement for antibiotics to maintain the plasmid propagation and Cas expression. Thus, there is a need to develop a generic type I CRISPR-Cas-based genome-editing system that can be used in diverse microbial hosts.

Therefore, it is an object of the invention to provide a chromosomal integrated type I CRISPR-Cas system for programmable genome editing which can be applied in diverse bacterial strains with different genetic backgrounds.

It is another object to provide compositions and methods for genomic editing and gene regulation in one or more pathological bacteria associated with development and progression of bacterial infections and diseases.

### SUMMARY OF THE INVENTION

A transferable genome-integrated type I-F CRISPR-Cas system for genomic modification of prokaryotic organisms has been developed. The transferable system can be integrated into the genomes of diverse bacterial strains having diverse genetic backgrounds, through site-specific recombination.

Compositions and methods for integrated and efficient genome editing of prokaryotic cells in a single step, using an editing plasmid are provided. Typically, the methods include one or more steps to integrate a ‘native’ type I-F cas operon including six cas genes (cas1, cas2-3, cas8f, cas5, cas7 and cas6) into a recipient bacterial cell genome at a highly conserved integration site. An exemplary recipient bacterial cell is a Pseudomonas aeruginosa cell. An exemplary type I-F cas operon is from Pseudomonas aeruginosa strain PA154197.

An exemplary highly conserved integration site is the attB integration site. In some embodiments, the methods include steps to enhance homologous recombination within the recipient cell. For example, in some embodiments the methods include one or more steps to incorporate the phage λ-red recombination system into the recipient cell.

In some embodiments, the methods include steps for detecting and confirming chromosomal integration within the recipient cell. For example, in some embodiments the methods include one or more steps to identify one or more reporter elements within the transferable systems. Preferably, the methods verify integration regardless of the un-conserved sequences flanking the integration site. An exemplary reporter element is a lacZ reporter gene. When a lacZ reporter is used, the methods identify chromosomal integration on the 5-bromo-4-chloro-3-indolyl-β-D-galactopyranoside (X-gal)-containing plates. Preferably, the reporter elements are driven by a strong promoter. An exemplary promoter is the Ptat promoter. In some embodiments, the integrated system contains one or more antibiotic resistance genes. An exemplary antibiotic resistance gene is the tetracycline-resistant gene.

In some embodiments, the methods include deleting, repressing or otherwise subduing the activity of an anti-CRISPR element within the recipient cell. In some embodiments, the methods include one or more steps to examine the activity of the CRISPR-Cas system in the recipient cell by introducing a targeting plasmid which encodes a crRNA complementary to the genomic element in the cell. In some embodiments, the targeted genomic element is the rhlI gene. An exemplary targeting plasmid encodes a crRNA targeting the rhlI gene is derived from the platform plasmid pPlatform (pAY5211) (Xu Z, et al., STAR Protocols 1, 100039, (2020)). In other embodiments, the targeted element is the Ptat promoter located upstream of the lacZ gene in the transferable system. An exemplary targeting plasmid targeting the Ptat promoter is a universal targeting plasmid pAY7138, which encodes a crRNA targeting to the Ptat promoter.

In some embodiments, the methods include one or more steps to edit the genome of the recipient cell by introducing one or more editing plasmids to the cell. Preferably, the integration of the transferable I-F cas system into the cell does not impact bacterial physiology compared with a control cell. For example, preferably the integration of transferable I-F cas system does not impact any of cell growth, proteolytic activity, biofilm formation, C. elegans killing, antibiotic susceptibility, colony morphology or motility in the recipient cell.

In other embodiments, the methods include one or more steps to integrate a transferable CRISPR-based transcriptional interference (transferable CRISPRi) system into a recipient bacterial cell genome. The methods provide site-specific binding of the Cascade to a genomic locus of interest preventing the recruitment or blocking the running of an RNA polymerase (RNAP) for transcription of a target gene in a microbial cell. The methods integrate cas genes for expression of multiple CRISPR-associated (Cas) proteins, including Cas1, Cas8f, Cas5, Cas7 and Cas6 within the microbial cell. In preferred embodiments, Cas2-3 gene is not present in a transferable CRISPRi system, thus lacking helicase and nuclease activities. Typically, integration of the transferable CRISPRi into the recipient cell includes contacting the cell with a transferable CRISPRi plasmid including one or more CRISPR RNA (crRNA) nucleic acids composed of individual spacers and flanking direct repeat sequence, which complex with Cas effector proteins and guide them to nucleic acid targets specific for the target gene within the recipient cell. Preferably, the one or more CRISPR RNA nucleic acids are configured to target one or more transcriptional sites of the gene of interest. Typically, the one or more transcriptional sites are selected from the group consisting of the RNA polymerase binding region, the transcription initiation region, the 5′-end of the coding region, the middle region of the gene, the 3′-end of the coding region, or a combination thereof, of the gene of interest in the recipient cell.

Typically, the type I-F CRISPR-Cas system has a targeting efficiency that is at least equivalent to that of the corresponding Cas9 system in an equivalent control cell. In a preferred embodiment, the type I-F CRISPR-Cas system has a targeting efficiency that is greater than that of the corresponding Cas9 system in an equivalent control cell.

The methods can efficiently edit and repress one or more target genes in the recipient cell. For example, in some embodiments, the methods induce from about 1%, 2%, 3%, 4%, 5%, 6%, 7%, 8%, 9%, 10%, 20%, 30%, 40%, 50%, 60%, 70%, 80%, 90%, up to 100% reduction in the expression or activity of a targeted gene in the recipient cell.

In some embodiments, the methods include steps to remove the integrated I-F cas system from the cell once genome editing has been achieved. Therefore, in some embodiments, the methods include one or more steps to ensure that only the selected modification in the recipient cell is carried out. In some embodiments, the methods remove the I-F cas system using a CRISPR-Cas removal plasmid. In an exemplary embodiment, the CRISPR-Cas removal plasmid encompasses a lacZ-targeting mini-CRISPR and a donor sequence upstream and downstream of homologous arms of the attB insertion site.

Compositions of nucleic acid vectors including the transferable I-F cas system are also provided. The compositions include a single vector including nucleic acids encoding the type I-F cas operon and one or more reporter genes configured for integration into the recipient microbial cell genome, and one or more crRNAs targeting a selected gene for editing on the same vector or on one or more additional nucleic acid vectors. In some embodiments, the compositions of nucleic acid vectors also include one or more targeting nucleic acid vectors to facilitate rapid analysis of CRISPR activity in a recipient cell. For example, in some embodiments, the targeting vector comprises crRNA targeting one or more reporter genes in the transferable I-F cas system.

Compositions of bacterial cells including the transferable I-F cas system are provided. The compositions and methods are particularly effective for manipulating the genome of Pseudomonas spp., such as P. aeruginosa. Therefore, in some embodiments, the bacterial cell including the transferable I-F cas system is a Pseudomonas spp. cell. In a preferred embodiment, the bacterial cell is a P. aeruginosa cell. An exemplary clinical isolate is a pathological P. aeruginosa strain. In some embodiments, the bacterial cell is a clinical isolate obtained from a subject. Exemplary strains of P. aeruginosa cell include strain PAO1, strain PA14, strain PA27853, strain PA150577, strain PA154197, strain PA151671, strain PA130788, and strain PA132533. In some embodiments, the bacterial cell contains an endogenous CRISPR-Cas system. In other embodiments, the bacterial cell does not contain an endogenous CRISPR-Cas system. In preferred embodiments, the bacterial cell does not contain anti-CRISPR elements that inactivate the transferable CRISPR-Cas system. In a particular embodiment, the bacterial cell is a P. aeruginosa strain PA130788 cell with the deletion of anti-CRISPR elements.

In some embodiments, the bacterial cell including the transferable I-F cas system is derived directly from the described methods to integrate a transferable I-F cas system into a recipient cell. In other embodiments, the cell is derived from a recipient cell produced according to the methods, for example a cell that is the product of reproduction of the cell produced according to the methods. Therefore, in some embodiments, the cell including the transferable I-F cas system is a product of 1 or more, up to 10, 20, 100 or 1,000 or more generations from the cell produced according to the described methods to integrate a transferable I-F cas system into a recipient cell.

Methods for characterizing the gene expression profile of a recipient bacterial cell following integration of the transferable I-F cas system have also been developed to assess the extent to which the cells are sensitive to gene manipulation through the type I-F CRISPR-Cas system. These methods are useful in the diagnosis, prognosis, selection of patients, and the treatment of bacterial diseases.

### BRIEF DESCRIPTION OF THE DRAWINGS

FIGS. 1A-1I show an overview of the transferable type I-F CRISPR-Cas system. FIG. 1A is a schematic showing gene architecture of the type I-F CRISPR-Cas system in PA154197. FIG. 1B is a cartoon representation of the transferable I-F cas system, indicating the locations of genes and main structures. The system carries a full I-F cas operon with its native promoter from PA154197, a lacZ reporter gene driven by the constitutive promoter Ptat, a fCTX integrase encoding gene int, a fCTX attachment site attP which recognize the chromosomal attB site, and two Flp recombinase target sites FRT. FIG. 1C is a diagram of the transferable λ-I-F cas system. An L-arabinose inducible λ-Red recombination system is assembled in the transferable I-F cas system. FIG. 1D is a diagram of the assembly of the targeting plasmid (pTargeting) and editing plasmid (pEditing) from the platform plasmid pAY5211. FIG. 1E is a schematic showing the transferable CRISPR-Cas-based genome editing in P. aeruginosa (PA). The transferable I-F cas system is first integrated into the attB site in the genome of a recipient strain. A targeting plasmid is transformed into the strain to examine the functionality of the CRISPR-Cas system. If the system is active, an editing plasmid is introduced to achieve desired genome editing. Recovered colonies are inoculated for luminescence-based screening. The luminescent inoculum is subjected to validation by PCR and Sanger sequencing. Once the editing is achieved, integrated I-F cas elements are removed by another round of editing using a CRISPR-Cas-removal plasmid. FIG. 1F is a photomicrograph image of X-gal-based selection for chromosomal CRISPR-integrated colonies. Blue colony indicates the expression of the integrated lacZ gene, meaning the successful chromosomal integration of the transferable cas system. FIG. 1G is a graph of whole genome sequencing result showing the site-specific integration of the transferable cas system. Arrow indicates no reads of integrated sequences in PAO1 WT at the attB site compared with PAO1^{λIF}. FIG. 1H is a graph showing quantitative PCR analysis of the expression of the integrated cas genes. FIG. 1I is bar chart showing self-targeting efficiency for each of the strains PAO1^{Ctrl}, PAO1^{IF}, PAO1^{λIF}, PAO1^{λIF}-20G, by introducing either pControl (pAY5211), or pTargeting (rhll), respectively. Single dots represent individual replicates (n=3 independent replicates).

FIGS. 2A-2F summarize data for self-targeting activity of the transferable type II Cas9 system in PAO1. FIG. 2A is a schematic representation of selected protospacers in the Ptat promoter and lacZ gene in PAO1^{λIF} and PAO1^{λCas9} strains for the self-targeting assay to examine the activities of two systems. Diamonds indicate the PAM location. Protospacers for type I-F system and Cas9 system are overlapped. CrRNAs in both strains target the same DNA strand. FIGS. 2B and 2C are bar charts showing self-targeting efficiency of the selected target sites shown in FIG. 2A in the Ptat promoter and lacZ gene for PAO1^{λIF} (FIG. 2B) and PAO1^{Cas9} (FIG. 2C) strains. FIG. 2D is a schematic representation of selected protospacers in the PrhlI promoter and rhlI gene in PAO1^{λIF} and PAO1^{λCas9} Strains for the Self-Targeting Assay to Examine the Activities of Two Systems. PAMs for both systems are located at same places. CrRNAs in two strains target to the different strands. FIGS. 2E and 2F are bar charts showing self-targeting efficiency of the selected target sites in the PrhlI promoter and rhlI gene for PAO1^{λIF} (FIG. 2E) and PAO1^{λCas9} (FIG. 2F) strains. Single dots represent individual replicates (n=3 independent replicates).

FIGS. 3A-3I show data demonstrating the transferable I-F CRISPR-Cas system enables precise gene deletion in the CRISPR-free strain PAO1. FIG. 3A shows the design and working mechanism of the editing plasmid pRhlI-Deletion-1 containing a rhlI-targeting mini-CRISPR and a donor template for rhlI deletion. The donor sequence (U+D) consisting of 833-bp upstream (U) and 805-bp downstream (D) homologous arms of the rhlI gene is shown. FIG. 3B is a photomicrograph of the result of rhlI deletion in the PAO1^{IF} strain. Primer pairs used to verify rhlI deletion in this study are indicated in FIG. 3A. Inoculum of each selected clone is shown. Loss of green color indicates the abolished PYO biosynthesis due to rhlI deletion. M: Marker. FIG. 3C is a photomicrograph of the result of rhlI deletion tested in 48 clones recovered from PAO^{IF}. FIG. 3D is a photomicrograph of the result of rhlI deletion indicated by the color of the bacterial inoculum. FIG. 3E is bar chart showing the efficiency of rhlI deletion using three different editing plasmids in PAO1^{IF} or PAO1^{λIF} treated with or without L-arabinose. FIG. 3F is a photomicrograph of the result of rhlI deletion in the PAO1^{λIF} strain without L-arabinose treatment. FIG. 3G is a photomicrograph of the result of rhlI deletion in the PAO1^{λIF} strain with L-arabinose treatment. FIG. 3H is a graph showing the result of whole genome sequencing results of the rhlI-deleted mutants and false positive clones. PAO1^{λIF} servers as the reference genome. FIG. 3I is a photomicrograph of the result of PCR examination of the rhlI deletion in PAO1. The amplified sequence flanking the attB site in PAO1 WT is 10.3 kb. No band was shown in lane 3 and 5 is due to the incapability of our PCR polymerase to amplify the large sequence containing the integrated 21.2-kb transferable λ-I-F cas system. The obtained PAO1 ΔrhlI mutant showed desired deletion of rhlI (lane 8) and removal of the transferred λ-I-F cas system (lane 7).

FIGS. 4A-4H show that transferable λ-I-F cas system enables gene deletion in different strains with distinct genetic background. FIG. 4A is a schematic showing genetic information about the presence or activity of native type I-F CRISPR-Cas systems in PAO1, PA14, PA150577, PA151671 and PA132533. FIG. 4B is a bar graph showing self-targeting efficiency for the PA14^{Ctrl} and PA14^{λIF} strains. FIG. 4C is a bar chart showing the editing efficiency of rhlI deletion in different PA14 derivatives. FIG. 4D is a diagram showing the plasmid pAYRecom designed for examining the recombination frequency of a host strain. pAYRecom contains two truncated Tc^{R} alleles separated by a gentamycin resistance cassette (Gen^{R}). When the plasmid is introduced into the host cell, recombination restores the functional Tc^{R} gene to confer tetracycline resistance. Thus, the recombination frequency can be quantified by the number of colonies recovered from tetracycline (recombinant recoveries) plates and kanamycin (total recoveries) plates. FIG. 4E shows the Tc^{R} recombinant frequency of PA14 which is ˜29% relative to that in PAO1. FIG. 4F is a bar graph showing self-targeting efficiency for the PA150577^{Ctrl} and PA150577 w strains. FIG. 4G is a photomicrograph of the result of rhlI deletion in PA150577^{λIF}, PA151671^{λIF} and PA132533^{λIF}. FIG. 4H shows the verification of the precise algR deletion in P. putida KT2440^{λIF} by colony PCR. Single dots represent individual replicates (n=3 independent replicates in FIGS. 4B and 4F, and n=2 in FIG. 4C).

FIGS. 5A-5E show that the presence of anti-CRISPR elements restricts genome editing. FIG. 5A is a schematic showing presence of an anti-CRISPR gene acr and its associated repressor gene aca in the PA130788 genome. FIG. 5B is a bar graph showing self-targeting efficiency for the PA130788^{λIF} stain with or without anti-CRISPR genes and overexpressed Aca protein. FIG. 5C is a photomicrograph of the result of rhlI deletion in PA130788^{λIF} Δacr/aca. FIG. 5D is a bar graph showing the relative expression of aca in PA130788^{λIF} and its derived strains measured by RT-qPCR. FIG. 5E is a bar graph showing the relative expression of acr in PA130788^{λIF} and its derived strains measured by RT-qPCR. Single dots represent individual replicates (n=3 independent replicates in FIG. 5B, and n=2 in FIGS. 5D and 5E).

FIGS. 6A-6G show transferable I-F CRISPRi-mediated gene repression. FIG. 6A is a diagram of the transferable I-F CRISPRi system. The system is derived from the transferable I-F cas system by removing the cas2-3 gene. A mini-CRISPR is assembled downstream of the Ptat promoter. FIG. 6B is a cartoon representation of the mechanism of CRISPRi-based gene repression. Site-specific binding of the Cascade to a genomic locus prevents the recruitment or blocks the running of an RNA polymerase (RNAP) for gene transcription. FIG. 6C is a schematic of the protospacers located at different loci in the PrhlI promoter and rhlI gene are selected to test the CRISPRi effect. Ps-1: −35 and −10 region; Ps-2: transcription initiation site; Ps-3: 5′-end of the rhlI coding region; Ps-4: middle region of the rhlI gene; Ps-5: 3′-end of the rhlI coding region. FIG. 6D is a bar graph showing effects of the transferable CRISPRi on the PYO production employing the targeting loci indicated in FIG. 6C in PA01. FIG. 6E is a bar graph showing effects of the transferable CRISPRi on the rhlI transcription employing the targeting loci indicated in FIG. 6C in PA27853 ΔacrF. FIG. 6F is a bar graph showing effects of the transferable CRISPRi on the PYO production employing the targeting loci indicated in FIG. 6C in PAO1. FIG. 6G is a bar graph showing effects of the transferable CRISPRi on the rhlI transcription employing the targeting loci indicated in FIG. 6C in PA27853 ΔacrF. Single dots represent individual replicates (n=3 independent replicates).

FIGS. 7A-7H show integration of the transferable elements has no significant effect on bacterial physiology. Comparison of the bacterial growth (FIG. 7A), proteolytic activity (FIG. 7B), biofilm formation (FIG. 7C), C. elegans killing ability (FIG. 7D), antibiotic resistance (FIG. 7E), colony biofilm morphology (FIG. 7F), motility (FIG. 7G), and transcriptome (FIG. 7H) between the PAO1 wild-type (PAO1 WT) and transferable CRISPR-Cas integrated (PAO1^{IF}) strains. (FIG. 7I) A gene PA1137 revealed by the RNA-seq result showing increased expression level in the PAO1¹ strain is confirmed by RT-qPCR. Single dots represent individual replicates (n=3 independent replicates).

FIGS. 8A and 8B show rhlI-targeting mini-CRISPR. FIG. 8A is a diagram showing the mini-CRISPR which encoding a crRNA that can target the rhlI gene in PAO1. FIG. 8B is a diagram showing the mini-CRISPR sequence.

FIGS. 9A-9D show integration and activity of the transferable λ-I-F cas system in different P. aeruginosa recipient strains. FIG. 9A showing the ordinary expression of the cas2-3 and cas7 genes in all the 30 transferred P. aeruginosa strains. Strains indicated by stars showed active self-targeting. Strains indicated by diamonds were whole genome sequenced. FIG. 9B is a schematic showing the universal targeting plasmid pAY7138 that expresses a crRNA complementary to a locus in the Ptat region upstream of the lacZ gene in the transferable system. FIG. 9C is a photomicrograph of the result of self-targeting assay showing the efficient targeting effect of pAY7138 in PAO1^{λIF}. FIG. 9D is a bar graph showing self-targeting efficiency for 10 clinical strains with active transferred λ-I-F cas system. Self-targeting results of the model stains PAO1, PA14 and an anti-CRISPR-containing strain PA130788 are also shown.

FIGS. 10A-10D show data assessing the activity of the transferable type II λ-Cas9 system in PAO1^{λCas9}. FIG. 10A is a diagram of the transferable type II λ-Cas9 system. FIG. 10B is a bar graph of RT-qPCR showing the expression of the cas9 and λ-red genes. FIG. 10C is a schematic showing the universal targeting plasmid pAY7149 that expresses a crRNA complementary to a locus in the Ptat region upstream of the lacZ gene in the transferable system. FIG. 10D is a photomicrograph of the result of self-targeting assay showing the inefficient targeting effect of pAY7149 in PAO1^{λCas9}

FIGS. 11A-11K show the data for harnessing the transferable type I-F cas system to achieve various genetic manipulations in PAO1. FIG. 11A is a bar graph showing expression of the λ-red genes in PAO1^{λIF} with treatment of different concentrations of L-arabinose. FIG. 11B is a photomicrograph of the result of a representative plate showing the screening for CRISPR-Cas-removed strains (white clones). Two white clones were selected and streaked onto X-gal (40 μg/ml) and tetracycline (Tet, 50 μg/ml)-containing plates. FIG. 11C is a photomicrograph of the result of PCR showing the amplified sequence flanking the attB site (10.3 kb in PAO1 WT). FIG. 11D is a photomicrograph of the result of curing of the editing plasmid in the edited cells. Edited cells could not grow in the presence of kanamycin (100 μg/ml) after plasmid curing. FIG. 11E are schematics showing the design of editing plasmids pRhlI-Deletion-1 and pRhlI-Deletion-2. FIG. 11F is a schematic showing the design of editing plasmids pRhlI-Deletion-3. FIG. 11G is a schematic showing the design of editing plasmids for N-terminal FLAG-tagging in mexF. A 32-bp sequence spanning the start codon (ATG) of mexF is selected as the protospacer and the donor contains the 24-bp FLAG sequence which immediately follows the start codon. FIG. 11H is a schematic showing the design of editing plasmids for C-terminal gfp-tagging in rhlA. A 32-bp sequence spanning the stop codon (TGA) of rhlA is selected as the protospacer and the donor contains the 714-bp gfp-encoding sequence which immediately upstream of the stop codon. Blue arrows indicate the primers used for verification. FIG. 11I is a photomicrograph of the results of FLAG-tagging in mexF and gfp-tagging in rhlA in the PAO1a strain. Primers used for PCR verification are indicated by the blue arrows in FIGS. 11G and 11H. Green fluorescence is detected in the clones with correct gfp insertion. FIG. 11J is a schematic showing the design of editing plasmid for point mutation in rhlI. The second cytosine (C54) in a PAM sequence “5′-C53C54-3′” is selected to conduct C to T substitution. The donor sequence in pRhlI-Deletion-1 (FIG. 8A) is replaced by another 500-bp donor sequence which spans the target site and contains the desired C54T substitution, generating the editing plasmid pRhlI-point. FIG. 11K is a representative DNA sequencing result showing the successful point mutation (C54T) in rhlI. The PAM sequence is framed by purple box.

FIGS. 12A-12B are charts showing the comparison of the I-F CRISPR-Cas systems from PA14 (FIG. 12A) and PA150577 (FIG. 12B) with PA154197.

FIGS. 13A-13D are bar charts showing transferable I-F CRISPRi-mediated gene repression in PA154197 Acas2-3 and PA153301 strains. FIG. 13A shows effects of the transferable CRISPRi on the rhlI transcription employing the targeting loci indicated in FIG. 6C in PA154197 Acas2-3. FIG. 13B shows effects of the transferable CRISPRi on the PYO production employing the targeting loci indicated in FIG. 6C in PA154197 Acas2-3. FIG. 13C shows effects of the transferable CRISPRi on the rhlI transcription employing the targeting loci indicated in FIG. 6C in PA153301. FIG. 13D shows effects of the transferable CRISPRi on the PYO production employing the targeting loci indicated in FIG. 6C in PA153301.

FIGS. 14A-14B show strategy and data for plasmid-based CRISPRi and its gene repression effect in PA154197 Acas2-3. FIG. 14A is a diagram showing the two plasmids used in the plasmid-based CRISPRi strategy. One is the transferable plasmid containing the type I-F cas system but without the cas2-3 gene. The other one is the targeting plasmid that express crRNA for site-specific targeting. FIG. 14B is a bar graph showing effects on the PYO production of plasmid-based CRISPRi at indicated loci in FIG. 6C in PA154197 Acas2-3.

FIGS. 15A-15B are nucleic acid sequencing data graphs showing the loss of spacers in the pqsA-targeting mini-CRISPR in the false positive clones. FIG. 15A shows Sanger sequencing data of mini-CRISPRs from eight false positive clones recovered from transformation of the pqsA-deleting plasmid. Their sequences were aligned with the designed pqsA-targeting mini-CRISPR. Loss of the spacer and one repeat sequence was found in two of the eight mini-CRISPRs. FIG. 15B shows sequence information for the designed mini-CRISPR for pqsA targeting. Framed sequences indicate the identical sequences flanking the mini-CRISPR in the plasmid.

FIGS. 16A-16C are photomicrographs show analysis of Cas9-based genome editing in PA14 and PA154197. FIG. 16A is a photomicrograph of the result of two-plasmid Cas9 system-based gene (mexR) deletion in PAO1. FIG. 16B is a photomicrograph of the result of recovery of the PA14 cells by introducing the positive control (Ctrl) plasmid. FIG. 16C is a photomicrograph of the result of analysis of the transformation rate of the PA154197 cells by introducing the mexR targeting plasmid.

### DETAILED DESCRIPTION OF THE INVENTION

### I. Definitions

The term “transferable I-F cas system” refers to the vector carrying the I-F cas operon. The term “transferable I-F CRISPR-Cas system” refers to the system employing the transferable I-F cas system and editing plasmids for gene editing.

The term “recipient cell” refers to a microbial cell, such as a bacterium, which has been modified by incorporation of the transferable I-F cas system.

The terms “targeted gene,” “targeted genome,” or “targeted element” refers to a gene or genomic component within a recipient microbial cell, which has been selected for modification by the CRISPR-Cas system.

The terms “gene editing,” “genome modification,” and “gene manipulation” are used interchangeably and refer to selective and specific changes to one or more targeted genes within a recipient cell through programming of the CRISPR-Cas system within the cell. The editing or changing of a targeted gene or genome can include one or more of a deletion, knock-in, point mutation, or any combination thereof in one or more genes of the recipient cell. Therefore, the result of the gene editing may be down-regulation or upregulation of one or more genes or expressed gene products as compared to a control cell without CRISPR-Cas-based gene editing. The extent of variation in the presence or activity of a gene or expressed gene product may be complete (i.e., 100%) or partial (i.e., 1-99.9%) of the level of that in a control cell. The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be measured as a % value, e.g., from 1% up to 100%, such as 5%, 10, 25, 50, 75, 80, 85, 90, 95, 99, or 100%. For example, gene repression or deletion may inhibit or reduce the activity and/or expression of one or more target genes, or the activity or quantity of one or more expressed gene products by about 10%, 20%, 30%, 40%, 50%, 75%, 85%, 90%, 95%, or 100% from the activity and/or quantity of the same gene or gene product in a control cell that is not subjected to CRISPR-Cas-base gene editing. In some embodiments, the inhibition and reduction are compared according to the level of mRNAs, or proteins corresponding to the targeted genetic element within the cell.

The terms “individual,” “subject,” and “patient” are used interchangeably, and refer to a mammal, including, but not limited to, murines, simians, humans, mammalian farm animals, mammalian sport animals, and mammalian pets.

The term “pharmaceutically acceptable” or “biocompatible” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The terms “treating” or “preventing” mean to ameliorate, reduce or otherwise stop a disease, disorder or condition from occurring or progressing in a subject which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition. Desirable effects of treatment include decreasing the rate of disease progression, ameliorating or palliating the disease state, and remission or improved prognosis. For example, an individual is successfully “treated” if one or more symptoms associated with a bacterial infection or associated disease or disorder are mitigated or eliminated, including, but are not limited to, reducing and/or inhibiting rate of bacterial cell proliferation/growth, increasing the quality of life of those suffering from the disease, decreasing the dose of medications required to treat the disease, delaying the progression of the disease, and/or prolonging survival of individuals.

### II. Compositions

A generic, transferable type I CRISPR-Cas-based genome-editing system that can be used in microbial hosts with diverse genetic backgrounds has been established. The system includes two or more nucleic acid vectors for expression of Cas genes and crRNA within a recipient host cell. The recipient microbial cell has a non-specific genetic background, and the type I CRISPR-Cas-based genome-editing system can integrate into the recipient cell genome in a single step, without the need for modification.

Class 1 CRISPR-Cas-based systems employ a multi-subunit effector complex which is known as the CRISPR-ASsociated Complex for Antiviral DEfense (“CASCADE”) to interfere with DNA or RNA within a cell. Class 1 systems are dependent on a small CRISPR RNAs (crRNA) to achieve site-specific DNA targeting and interference of genomic DNA.

Compositions for a chromosomal integrated type I-F cas system for programmable genome editing and robust gene regulation are provided. The chromosomal integration system overcomes the limitations of narrow host range and the requirement for antibiotics to maintain its propagation and cas expression, which are associated with plasmid-encoded Cas proteins.

In some embodiments, the compositions are effective to selectively and specifically edit and/or regulate the genome of multiple microbial species with diverse genotypes. The compositions are particularly effective for gene editing and/or gene regulation in multiple different Pseudomonas spp., such as strains of P. aeruginosa. In some embodiments, the compositions are effective to selectively and specifically edit and/or regulate the genome of multiple microbial species, particularly different Pseudomonas spp., with or without their own native CRISPR-Cas systems, with and without available genome sequences, with and without anti-CRISPR systems. In other embodiments, the compositions are effective for gene editing and/or gene regulation in Acinetobacter baumannii.

The Cas9-mediated genome editing system requires consecutive transformation of two plasmids with two different antibiotic markers. However, most P. aeruginosa strains, especially those wild isolates with clinical and environmental significance, have a poor DNA homeostasis capacity and are not tolerant to transformation and maintenance of two different plasmids. Hence, thus far, success of the Cas9-based genome editing is only limited to the model strains PAO1 and PAK. P. aeruginosa naturally harbors the type I-F CRISPR-Cas system in many of its genomes. In preferred embodiments, the compositions achieve high efficiency in PAO1 with much simpler procedures than those of Cas9-based method. In further preferred embodiments, the compositions are effective to selectively and specifically edit and/or regulate the genome of those clinical and environmental isolates of Pseudomonas spp., where the Cas9-based method is not applicable.

The Cas9 and Cas12a are generally derived from Streptococcus pyogenes or Lachnospiraceae bacterium, and need to be over-expressed when being exploited for genome editing in heterologous hosts. In preferred embodiments, the transferrable type I-F CRISPR-Cas system is expressed from its native promoter when the system is transferred to heterologous hosts. In further preferred embodiments, the transferrable type I-F CRISPR-Cas system is derived from a clinical P. aeruginosa strain PA154197.

Preferably, the compositions cause little to no intrinsic toxicity in the hosts. For example, the integration of the transferable I-F cas system into the cell does not impact bacterial physiology compared with a control cell. For example, preferably the integration of transferable I-F cas system does not impact any of cell growth, proteolytic activity, biofilm formation, C. elegans killing, antibiotic susceptibility, colony morphology or motility in the recipient cell.

A recent study reported repurposed the type I-F CRISPR-Cas system for transcriptional activation in human HEK293T cells (Chen et al., Nat Commun, 2020,11:3136). Thus, in some embodiments, the compositions are also effective to selectively and specifically edit and/or regulate the genome of eukaryotic cells.

A. Nucleic Acid Vectors

Nucleic acid vectors for changing, adding or deleting one or more genes in a microbial cell are described. Typically, two specific vectors are required. A first, type I-F cas system nucleic acid vector, integrates a functional type I-F cas operon into the genome of a recipient microbial cell. Once the transferable system is integrated into the genome of a recipient strain, it can be stably expressed in stoichiometry and enables precise and rapid genome editing using a single editing plasmid containing a pre-designed CRISPR RNA (crRNA)-expressing mini-CRISPR element and repair donor in one-step.

1. Type I-F cas System Vector

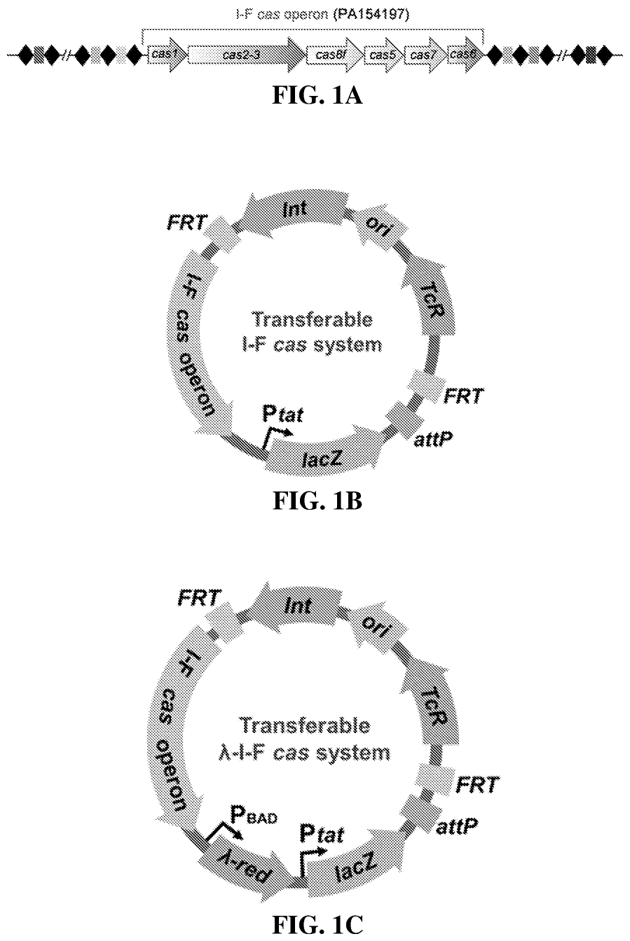

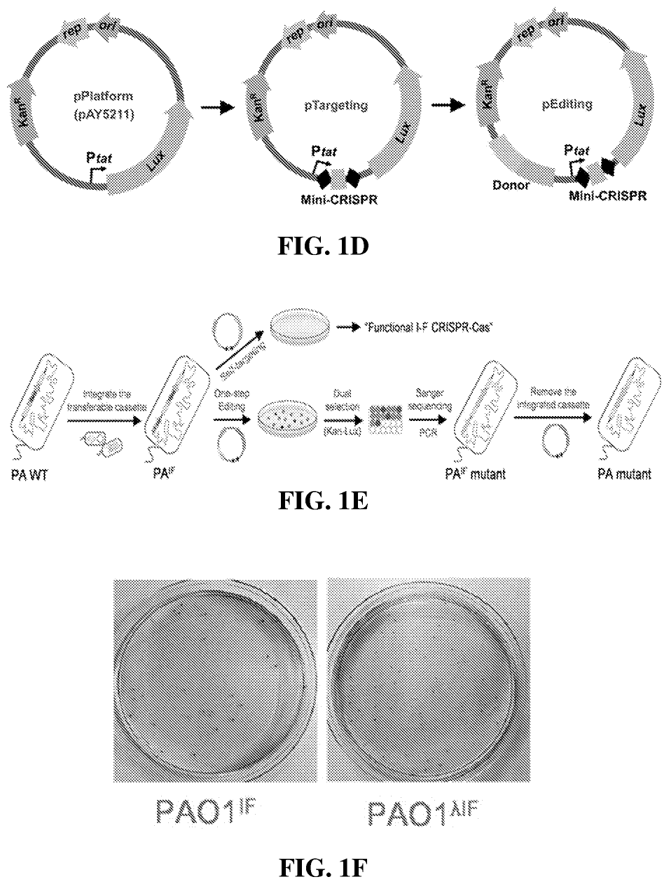

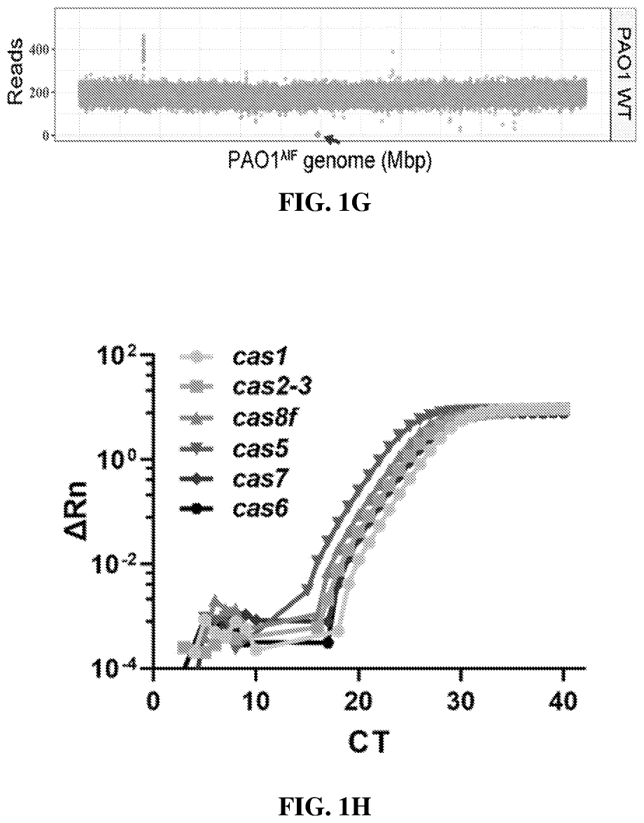

A type I-F cas system nucleic acid vector includes nucleic acids configured to integrate a functional type I-F cas operon into the genome of a microbial cell. In some embodiments, the microbial cell is a Pseudomonas bacterium. Upon integration of the type I-F cas system into a recipient microbial cell, gene editing can be carried out in a single step, by application of a nucleic acid editing vector, including one or more CRISPR RNAs (crRNAs) targeting one or more selected genes for insertion, deletion, or modification within the microbial cell genome. Typically, the Type I-F cas system vector is a nucleic acid plasmid, including nucleic acids configured to include a type I-F cas operon, one or more elements for integration of the cas operon into the genome of the recipient microbial cell, and one or more reporter genes for assessing integration and CRISPR-Cas expression/activity. A schematic representation of the Type I-F Cas system vector is set forth in FIG. 1B.

In some embodiments, the transferable system enables integration of the entire cas gene cluster (8.693 kb) including the cas genes cas1, cas2-3, cas8f, cas5, cas7 and cas6 into the genome of the strain of interest, thus enabling the stable expression of six cas genes in heterologous bacterial hosts. In preferred embodiments, the genome-editing process involves a one-step transformation of a single editing plasmid. Thus, compared to the time-consuming methods currently used in Pseudomonas species such as the counter selection-based method and the two-plasmid-based Cas9 method, the transferable system thus generates desired mutants with efficiency and simplicity.

a. I-F Cas Operon

Nucleic acid vectors including nucleic acids configured to integrate a transferable type I-F cas system vector into the genome of a microbial cell include the I-F cas operon with a native promoter. The operon includes the cas genes cas1, cas2-3, cas8f, cas5, cas7 and cas6, and a nucleic acid sequence configured to promote transcription of the cas genes in the microbial cell. A schematic representation of the I-F cas operon is set forth in FIG. 1A. In some embodiments, the I-F cas operon is from the highly active I-F CRISPR-Cas system in a clinically isolated multidrug-resistant Pseudomonas aeruginosa strain PA154197, which encompasses six cas genes sandwiched by two convergent CRISPR arrays. This system recognizes a canonical 32-bp protospacer preceded by a di-nucleotide protospacer adjacent motif (PAM) of 5′-CC-3′. Five Cas proteins, Cas8f (Csy1), Cas5 (Csy2), Cas7 (Csy3) and Cas6f (Csy4) which are assembled in the Cascade and Cas2-3 which contains helicase and nuclease domains, are involved in the target DNA recognition and cleavage under the guidance of a crRNA.

Where the DNA cleavage ability is not desired, the Cas2-3 gene is modified in the I-F cas operon such that its DNA cleavage ability is disabled. For example, when repression of a particular target gene is desired, upon the presence of a programmable crRNA, the Cascade complex targets a genomic locus specifically and stably, which prevents the recruitment or movement of the RNA polymerase (RNAP) and consequently inactivates the expression of the target gene. Thus, in some embodiments, Cas2-3 gene is eliminated from the I-F cas operon.

b. Elements for Genomic Integration

The vector includes one or more nucleic acid sequences configured for integration of the vector into the genome of the recipient microbial cell, at a pre-determined region/site. The vector enables efficient integration of the type I-F cas operon from PA154197 into a conserved attB site which is present in diverse P. aeruginosa strains (Genomic location: 2,947,580-2,947,610 in strain PAO1). When the transferable systems are introduced into P. aeruginosa strains, they can integrate efficiently into the specific attB site, which is highly conserved among the P. aeruginosa species. The vector also includes nucleic acid sequences configured as one or more genes encoding an integrase enzyme; a nucleic acid sequence encoding an integration site configured to recognize and attach the plasmid to a target attachment site within the microbial genome.

c. Reporter Systems

The vector includes one or more nucleic acid sequences configured to be one or more reporter genes for expression within the recipient cell, and optionally one or more nucleic acid sequences configured to promote transcription of the reporter gene(s) upon integration into the microbial cell genome. In a preferred embodiment, A lacZ reporter gene, driven by a strong promoter is designed in the transferable system. In some embodiments, the strong promoter is the Ptat promoter. When a lacZ reporter gene is included, chromosomal integration of the system can be easily detected on the 5-bromo-4-chloro-3-indolyl-β-D-galactopyranoside (X-gal)-containing plates. For example, successful chromosomal integration and expression of the lacZ gene following introduction of the transferable systems into the recipient microbial cell is indicated by recovery of blue cells. Reporter systems, including antibiotic resistance genes, are known in the art.

d. λ-Red Recombination System

For strains with insufficient homologous recombination capacities, the integrative λ-red recombination system can be included within the Type I-F cas system vector to enable the simultaneous introduction of both the I-F Cascade and λ-red recombination system into the microbial cell. A functional phage λ-red recombination system includes genes encoding λ-red proteins Exo, Gam, and Beta, and an arabinose-inducible promoter. Therefore, in some embodiments, the vector also includes nucleic acids configured to express the genes Exo, Gam, and Beta of the λ-red recombination system and an arabinose-inducible promoter. A schematic representation of the Type I-F cas system vector, including the λ-red recombination system is set forth in FIG. 1C.

2. crRNA Expression Vectors

Nucleic acid vectors to enable specific, designed genome editing of the microbial recipient cell, including gene deletion, gene knock-in, point mutation, and/or gene modification via a one-step procedure through incorporation of one or more CRISPR RNAs (crRNAs) within the recipient microbial cell are also described. In some embodiments, a crRNA expression vector is configured to include crRNA, which, upon expression within the recipient microbial cell, enables assessment of the efficiency and/or activity of the Type I-F cas system within the recipient cell. In other embodiments, a crRNA expressing vector removes the Type I-F cas system from the recipient cell, for example, following gene editing, to prevent subsequent CRISPR-Cas activity within the cell.

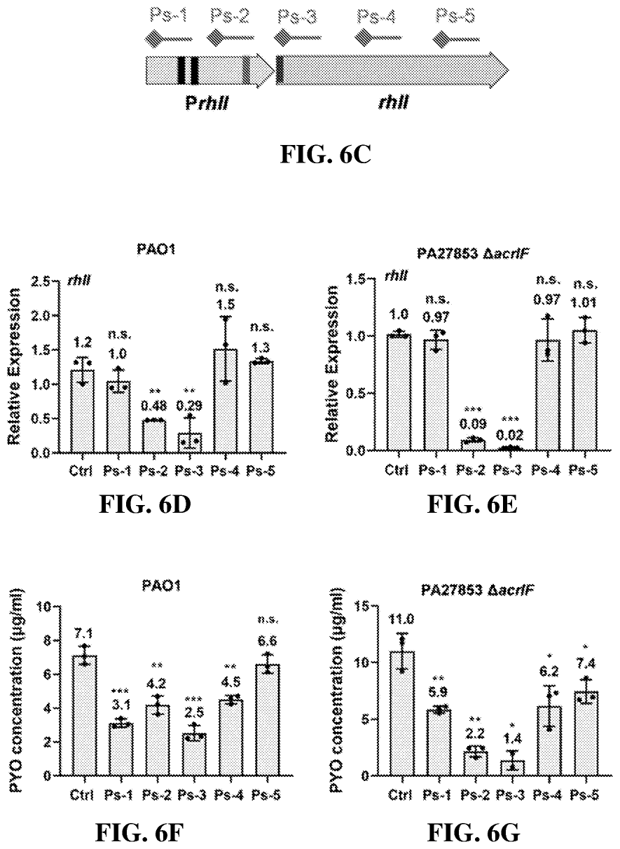

In other embodiments, the one or more crRNAs are on the same plasmid as the I-F cas system. For example, a programmable mini-CRISPR insertion site downstream of the Ptat promoter was designed in the transferable system so that crRNA can be constitutively expressed from the chromosome to execute robust Cascade targeting as shown in FIG. 6A. In some embodiments, the one or more crRNA nucleic acids are configured to target one or more transcriptional sites of the gene of interest, including the RNA polymerase binding region, the transcription initiation region, the 5′-end of the coding region, the middle region of the gene, the 3′-end of the coding region, or a combination thereof.

a. CRISPR-Cas Targeting Vectors

In some embodiments, the crRNA expression vector is configured to express one or more CRISPR RNAs (crRNAs) designed for assessing the efficiency and/or activity of a Cas operon (“CRISPR-Cas Targeting Vectors”) within a recipient microbial cell. Typically, vectors for assessing the efficiency and/or activity of a Cas operon within a recipient cell target the one or more reporter genes (selection markers) within the transferable type I-F cas system. Assessing the efficacy of the type I-F CRISPR-Cas system within the recipient cell is then facilitated by assessing the recovered survival cells by introducing the targeting vector. For example, Interference activities of the transferred cas systems in all recipient strains are examined by comparing the relative conjugation efficiency between a microbial cell receiving a control plasmid, and an active Targeting vector. Recipient cell strains showing active interference in the presence of the transferred cas system have active genome editing capabilities. A schematic representation of a Targeting vector is provided in FIG. 1D.

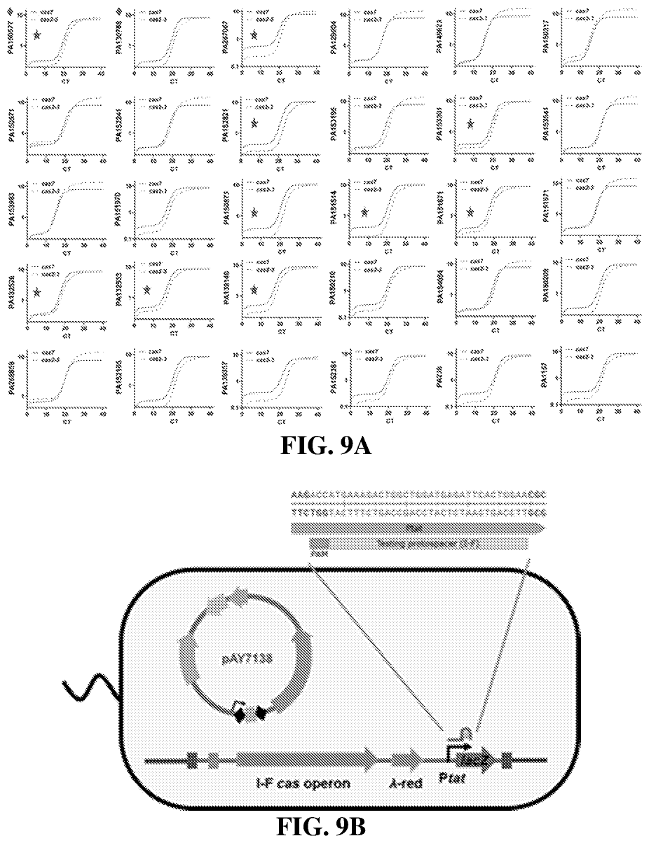

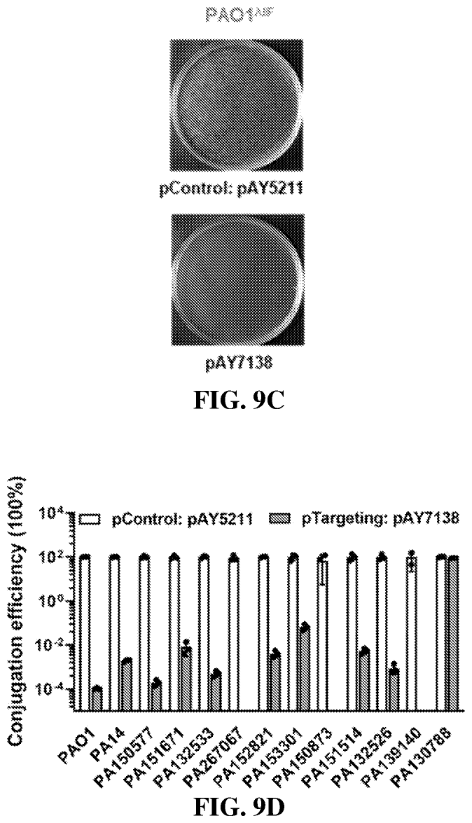

In some embodiments, the targeting vector comprises crRNA nucleic acids configured to disrupt one or more genes associated with expression of the Acyl-homoserine-lactone synthase enzyme. For example, in some embodiments, the Targeting vector includes nucleic acids configured to express a crRNA targeting the rhlI gene in the PAO1 strain (“pRhlI-Targeting”). An exemplary plasmid pRhlI-Targeting is constructed based on the platform plasmid pPlatform (pAY5211) (Xu & Yan, STAR Protocols 1, 100039, (2020)). The mini-CRISPR in pRhlI-Targeting encompasses a 32-bp spacer that is complementary to a “5′-CC-3′”-preceded protospacers within the rhlI gene in PAO1 (FIG. 8A). Therefore, introduction of the pRhlI-Targeting plasmid in PAO1 will induce significantly decreased efficiency of conjugation. A reduced conjugation efficiency relative to a control plasmid indicates that the transferred I-F cas system in the recipient cell is highly active to execute genome cleavage. In other embodiments, the Targeting vector includes nucleic acids configured to encode a crRNA targeting to the Ptat promoter, located upstream of the lacZ gene in the transferable system (FIG. 9B). In particular embodiments, the Targeting vector is a universal targeting plasmid pAY7138, which encodes a crRNA targeting to the Ptat promoter.

b. Designed Genetic Editing Vectors

In some embodiments, the crRNA vector is configured to express one or more crRNAs and carry repair donor(s) designed for genetic modification at one or more selected genomic regions (“Genetic editing vector”) within the microbial cell through the Type I-F CRISPR-Cas system. Gene editing nucleic acid vectors include nucleic acids configured to express one or more crRNA nucleic acids and repair donor(s) configured to change, add or delete one or more target genomic sites in the microbial cell. A schematic representation of an Editing vector is provided in FIG. 1D.

c. CRISPR-Cas Removal Vectors

In some embodiments, the crRNA expression vector is configured to express one or more crRNAs and carry a donor specific for removal of the integrated type I-F cas system from the recipient cell (“Removal vector”). After the desired genome editing is achieved, the transferred system can be conveniently removed from the genome using a Removal vector, in combination with one or more selection systems. Therefore, in some embodiments, the crRNA expression vector is a Removal vector, comprising one or more crRNA nucleic acids configured to disrupt one or more target genomic sites within the type I-F cas system that has been integrated into the genome of the recipient microbial cell.

3. Control Vectors

The nucleic acid vectors can also be control vectors, which lack one or more of the nucleic acid sequences required for the activity of the Type I-F Cas System Vector, or one or more of the crRNA expression vectors. Therefore, the activity of any of the components of the Type I-F Cas System within a recipient bacterial cell can be compared to a control cell, which lacks the component. In some embodiments, the presence or lack of one or more specific CRISPR-Cas components within a recipient cell is visualized by inspection of one or more expressed gene products of a reporter gene.

B. Recipient Microbial Cells

The transferable system is designed for integration into microbial/bacterial cells having distinct genomes, therefore the microbial host cells can have diverse genetic backgrounds. Microbial host cells including the transferable type I-F cas system integrated within the cell's genome are described. In some embodiments, the recombinant recipient cell includes a recombinant nucleic acid type I-F cas system including (i) a type I-F cas operon, including the cas genes cas1, cas2-3, cas8f, cas5, cas7 and cas6 and a nucleic acid sequence configured to promote transcription of the cas genes in the microbial cell; (ii) one or more genes encoding an integrase enzyme; (iii) a nucleic acid sequence encoding an integration site configured to recognize and attach the plasmid to a target attachment site within the microbial genome; (iv) two nucleic acid sequences configured to be an Flp recombinase target sites; and (v) one or more reporter genes and a nucleic acid sequence configured to promote transcription of the reporter gene(s) upon integration into the microbial cell genome. The vector is integrated into the genome of the microbial cell via attachment at the target attachment site, such as the conserved attB site.

In some embodiments, the strain and/or genotype of the host cell is unknown. In some embodiments, the recipient cells are part of a population of genetically distinct strains of bacteria, for example, a group of two or more genetically different strains of P. aeruginosa.

1. Pseudomonas Spp.

In a preferred embodiment, the microbial host cell is a Pseudomonas spp. bacterium. In a more preferred embodiment, wherein the cell is a P. aeruginosa bacterium. In some embodiments, the microbial cell is a P. aeruginosa bacterium strain selected from strain PAO1, strain PA14, strain PA27853, strain PA150577, strain PA154197, strain PA151671, strain PA130788, and strain PA132533. In some embodiments, the recipient microbial cell is a P. aeruginosa strain bacterium, wherein endogenous anti-CRISPR elements have been disrupted. In a particular embodiment, the recipient cell is a P. aeruginosa strain PA130788 cell.

Many Pseudomonas spp. cells include functional endogenous CRISPR-Cas systems, and exhibit endogenous CRISPR-Cas activity. However, many Pseudomonas spp. cells contain non-functional or semi-functional CRISPR-Cas systems, or do not possess any endogenous CRISPR-Cas activity. Pseudomonas spp. cells including endogenous CRISPR-Cas activity, as well as those lacking functional endogenous activity may be recipient cells of the described transferable I-F cas system. Therefore, in some embodiments, the recipient microbial cell does not contain an endogenous CRISPR-Cas system. In other embodiments, the recipient Pseudomonas spp. cell contains an endogenous CRISPR-Cas system which is functional, or which is fully or partially non-functional. In some embodiments, the recipient Pseudomonas spp. cell contains genes encoding an endogenous CRISPR-Cas system which is functional, but the functionality is precluded, reduced or minimized by the presence of one or more endogenous anti-CRISPR genes, or anti-CRISPR elements within the cell.

### III. Methods of Genetic Modification

Methods for genome editing of recipient host cells using a transferable I-F CRISPR-Cas system have been developed. Methods for changing, adding or deleting one or more genes in a microbial cell are provided. In certain embodiments, the methods include administering to a bacterial cell a vector including a transferable I-F cas system in combination with one or more nucleic acid editing plasmids including one or more crRNAs and repair donors designed to change, add or delete one or more genes in a microbial cell. Methods for construction, reproduction, amplification, and conjugation of the recipient cells are also described. In particular, the transferable I-F cas system is passed from one donor Escherichia coli SM10 strain to the recipient strain through bacterial conjugation.

A. I-F CRISPR-Cas-Mediated Genetic Modification

Methods for changing, adding or deleting one or more genes in a bacterial cell are described. In some embodiments, the methods reduce, minimize or abrogate the expression or activity of a target gene in the recipient cell. For example, in some embodiments, the methods induce from about 1%, 2%, 3%, 4%, 5%, 6%, 7%, 8%, 9%, 10%, 20%, 30%, 40%, 50%, 60%, 70%, 80%, 90%, up to 100% reduction in the transcription or activity of a target gene in the recipient cell.

Methods for I-F CRISPR-Cas-mediated genetic modification of a microbial cell include the steps of:
(a) transforming a bacterial cell with a nucleic acid type I-F cas system vector, including (i) a type I-F cas operon, comprising the cas genes cas1, cas2-3, cas8f, cas5, cas7 and cas6 and a nucleic acid sequence configured to promote transcription of the cas genes in the microbial cell; (ii) one or more genes encoding an integrase enzyme; (iii) a nucleic acid sequence encoding an integration site configured to recognize and attach the plasmid to a target attachment site within the microbial genome; (iv) two nucleic acid sequences configured to be an Flp recombinase target site; and (v) one or more reporter genes and a nucleic acid sequence configured to promote transcription of the reporter gene(s) upon integration into the bacterial cell genome. The contacting occurs under conditions suitable for integration of the type I-F cas system into the bacterial cell genome to form a recipient cell.

In some embodiments, the nucleic acid type I-F cas system vector further includes a functional phage λ-red recombination system, including genes encoding λ-Red proteins Exo, Gam, and Beta, and an arabinose-inducible promoter.

The transformation of cells can include any specific techniques for transformation of microbial cells with nucleic acids known in the art, including bacterial conjugation, whereby a plasmid is transferred from one bacterium to another. In a preferred embodiment, an E. coli SM10 strain including the type I-F cas system vector is used as a donor cell to transfer the type I-F cas system vector to a P. aeruginosa recipient cell by bacterial conjugation.

Chromosomal integration of the type I-F cas system having a lacZ reporter gene can be easily detected on the 5-bromo-4-chloro-3-indolyl-β-D-galactopyranoside (X-gal)-containing plates: the presence of blue colonies in the recipient cells transformed with the type I-F cas system vector indicates successful integration in the recipient cells.
(b) transforming the recipient cell with a nucleic acid editing vector, including one or more CRISPR RNA (crRNA) nucleic acids and repair donor(s), configured to change, add or deleting one or more target genomic sites in the recipient cell through the type I-F CRISPR-Cas system.

The methods can include the optional step to assess the activity of the transferred type I-F cas system in the recipient cell. Therefore, in some embodiments, the methods include the step of:
(c) assessing the self-targeting activity by introducing the recipient cell with a nucleic acid Targeting vector, comprising one or more CRISPR RNA (crRNA) nucleic acids configured to disrupt one or more target genomic sites in the microbial cell genome or the type I-F cas system. In particular embodiments, the methods include expressing within the recipient cell one or more CRISPR RNA (crRNA) nucleic acids configured to disrupt the rhlI gene, or the Ptat promoter, or both, for assessment of the activity of the type I-F CRISPR-Cas system. Therefore, in some embodiments, the methods include transforming the recipient cell with a crRNA nucleic acid targeting vector including one or more CRISPR RNA (crRNA) nucleic acids configured to disrupt the rhlI gene, or the Ptat promoter, or both.

In some embodiments, the methods further include the step of:
(d) removing the type I-F cas system from the recipient cell by transforming the recipient cell with a nucleic acid CRISPR-Cas removal vector. The methods optionally include a step of removing the type I-F cas system from a recipient cell, for example, following successful completion of gene editing. Removal of the type I-F cas system prevents any further CRISPR-Cas mediated gene editing within the recipient cell, and therefore preserves the fidelity of the desired genetic modification with the cell. Therefore, in some embodiments, the methods include transforming the recipient cell with a CRISPR-Cas Removal vector, including one or more nucleic acids configured to be a lacZ-targeting CRISPR RNA (crRNA), for example, a lacZ-targeting mini-CRISPR and a donor sequence upstream and downstream of homologous arms of the attB insertion site.

B. Methods for Gene Repression

Methods for repressing a gene of interest in a bacterial cell are described. In some embodiments, the methods reduce, minimize or abrogate the transcription of a target gene in the recipient cell. For example, in some embodiments, the methods induce from about 1%, 2%, 3%, 4%, 5%, 6%, 7%, 8%, 9%, 10%, 20%, 30%, 40%, 50%, 60%, 70%, 80%, 90%, up to 100% reduction in the transcription of a target gene in the recipient cell.

In some embodiments, repression of a particular target gene is desired, the cas2-3 gene is eliminated from the I-F cas operon. Thus, upon the presence of a programmable crRNA, the Cascade complex targets a genomic locus specifically and stably, which prevents the recruitment or movement of the RNA polymerase (RNAP) and consequently inactivates the expression of the gene of interest. Thus, in some embodiments, the cas2-3 gene is modified in the I-F cas operon such that its DNA cleavage ability is disabled.

Methods for introducing a transferable CRISPR-based transcriptional interference (transferable CRISPRi) system in a microbial cell include the steps of:
(a) transforming a bacterial cell with a nucleic acid type I-F CRISPRi system vector, including (i) a type I-F cas operon, comprising the cas genes cas1, cas8f, cas5, cas7 and cas6 and a nucleic acid sequence configured to promote transcription of the cas genes in the microbial cell; (ii) one or more genes encoding an integrase enzyme; (iii) a nucleic acid sequence encoding an integration site configured to recognize and attach the plasmid to a target attachment site within the microbial genome; (iv) two nucleic acid sequences configured to be an Flp recombinase target site; (v) one or more reporter genes and a nucleic acid sequence configured to promote transcription of the reporter gene(s) upon integration into the bacterial cell genome, and (vi) one or more CRISPR RNA (crRNA)-encoding nucleic acids, configured to target one or more sites of the gene of interest in the microbial cell through the type I-F CRISPRi system. The contacting occurs under conditions suitable for integration of the type I-F CRISPRi system into the bacterial cell genome to form a recipient cell.

In some embodiments, the one or more CRISPR RNA nucleic acids are configured to target one or more transcriptional sites of the gene of interest, including the RNA polymerase binding region, the transcription initiation region, the 5′-end of the coding region, the middle region of the gene, the 3′-end of the coding region, or a combination thereof.

The transformation of cells can include any specific techniques for transformation of microbial cells with nucleic acids known in the art, including bacterial conjugation, whereby a plasmid is transferred from one bacterium to another. In a preferred embodiment, an E. coli SM10 strain including the type I-F CRISPRi system vector is used as a donor cell to transfer the type I-F CRISPRi system vector to a P. aeruginosa recipient cell by bacterial conjugation.

Chromosomal integration of the type I-F CRISPRi system having a lacZ reporter gene can be easily detected on the 5-bromo-4-chloro-3-indolyl-β-D-galactopyranoside (λ-gal)-containing plates: the presence of blue colonies in the recipient cells transformed with the type I-F CRISPRi system vector indicates successful integration in the recipient cells.

### IV. Kits

Kits are also disclosed. The kits can include, for example, an aliquot of the type I-F cas system Vector, and at least one Editing Vector, or a combination thereof in separately or together in the same admixture. The active agents can be supplied alone (e.g., lyophilized), or in a mixture composition. The active agents can be in a unit amount for transformation into a microbial host cell, or in a stock that should be diluted prior to use. In some embodiments, the kit includes a supply of buffers and reagents required for transformation of a bacterial cell. In some embodiments, the kit includes the type I-F cas system Vector, and one or more of a Targeting Vector, an Editing Vector, and a Removal Vector. The kit can also include devices for use of the active agents or compositions, for example, donor E. coli cells including the type I-F cas system Vector, syringes and pipettes. The kits can include printed instructions for use of the reagents according to the methods described above.

The present invention is further understood by reference to the following non-limiting examples.

### EXAMPLES

### Example 1: Transferable I-F CRISPR-Cas System is Active in a Broad Host-Range of P. aeruginosa Strains

### Materials and Methods

Primers, Bacterial Strains, and Growth Conditions

Primers and bacterial strains used in this study are listed in Table 1. E. coli DH5α is used for plasmid propagation and is usually cultured at 37° C. in Luria-Bertani (LB) broth or on the LB agar plate supplemented with required antibiotics. E. coli SM10 is used for conjugative plasmid delivery. P. aeruginosa clinical strains were isolated from the Queen Mary Hospital in Hong Kong, China. Antibiotics supplemented in the agar plates for DH5α were 20 μg/ml kanamycin, 10 μg/ml tetracycline and 200 μg/ml ampicillin. Antibiotics supplemented in the agar plates for SM10 were 500 μg/ml kanamycin, 10 μg/ml tetracycline and 200 μg/ml ampicillin. Antibiotics supplemented in the agar plates for P. aeruginosa strains were 500 μg/ml kanamycin, 50 μg/ml tetracycline and 200 μg/ml carbenicillin.

TABLE 1
Bacterial strains, primers used in this study

| | | |
|---|---|---|
| Strains and primers | Description | Source |

Strains:

| | | |
|---|---|---|
| DH5α | F- endA1 glnV44 thi- | Lab collection |
| | 1 recA1 relA1 gyrA96 deoR nupG purB20 φ80dlacZΔM15 | |
| SM10 | Δ(lacZYA-argF)U169, hsdR17(r_{K}-m_{K+}), λ- | |
| | thi thr leu tonA lacY supE recA:RP4-2-Tc: Mu Km λpir | Lab collection |
| PAO1 | Pseudomonas aeruginosa laboratory strain | Lab collection |
| PA14 | Pseudomonas aeruginosa laboratory strain | Lab collection |
| PA154197 | PA clinical isolate from Queen Mary Hospital (HK) | Lab collection |
| PA27853 | PA clinical isolate from Queen Mary Hospital (HK) | Lab collection |
| PA150577 | PA clinical isolate from Queen Mary Hospital (HK) | Lab collection |
| PA130788 | PA clinical isolate from Queen Mary Hospital (HK) | Lab collection |
| PA267067 | PA clinical isolate from Queen Mary Hospital (HK) | Lab collection |
| PA129904 | PA clinical isolate from Queen Mary Hospital (HK) | Lab collection |
| PA149623 | PA clinical isolate from Queen Mary Hospital (HK) | Lab collection |
| PA150317 | PA clinical isolate from Queen Mary Hospital (HK) | Lab collection |
| PA150571 | PA clinical isolate from Queen Mary Hospital (HK) | Lab collection |
| PA152241 | PA clinical isolate from Queen Mary Hospital (HK) | Lab collection |
| PA152821 | PA clinical isolate from Queen Mary Hospital (HK) | Lab collection |
| PA153195 | PA clinical isolate from Queen Mary Hospital (HK) | Lab collection |
| PA153301 | PA clinical isolate from Queen Mary Hospital (HK) | Lab collection |
| PA153541 | PA clinical isolate from Queen Mary Hospital (HK) | Lab collection |
| PA153983 | PA clinical isolate from Queen Mary Hospital (HK) | Lab collection |
| PA151970 | PA clinical isolate from Queen Mary Hospital (HK) | Lab collection |
| PA150873 | PA clinical isolate from Queen Mary Hospital (HK) | Lab collection |
| PA151514 | PA clinical isolate from Queen Mary Hospital (HK) | Lab collection |
| PA151671 | PA clinical isolate from Queen Mary Hospital (HK) | Lab collection |
| PA151971 | PA clinical isolate from Queen Mary Hospital (HK) | Lab collection |
| PA132526 | PA clinical isolate from Queen Mary Hospital (HK) | Lab collection |
| PA132533 | PA clinical isolate from Queen Mary Hospital (HK) | Lab collection |
| PA139140 | PA clinical isolate from Queen Mary Hospital (HK) | Lab collection |
| PA150210 | PA clinical isolate from Queen Mary Hospital (HK) | Lab collection |
| PA154054 | PA clinical isolate from Queen Mary Hospital (HK) | Lab collection |
| PA150209 | PA clinical isolate from Queen Mary Hospital (HK) | Lab collection |
| PA268859 | PA clinical isolate from Queen Mary Hospital (HK) | Lab collection |
| PA152165 | PA clinical isolate from Queen Mary Hospital (HK) | Lab collection |
| PA139357 | PA clinical isolate from Queen Mary Hospital (HK) | Lab collection |
| PA152361 | PA clinical isolate from Queen Mary Hospital (HK) | Lab collection |
| PA238 | PA isolate from the surface layer of the North | Lab collection |
| | Pacific Ocean | |
| PA1157 | PA isolate from the surface layer of the North | Lab collection |
| | Pacific Ocean | |

Primers:

| | | |
|---|---|---|
| pUC57-F | AGGGTTTTCCCAGTCACGAC | This study |
| pUC57-R | AGCGGATAACAATTTCACAC | This study |
| pMS402-F | CCAGCTGGCAATTCCGA | This study |
| pMS402- R | AATCATCACTTTCGGGAAAG | This study |
| CTX-Kpn-IF-cas-F | CTATAGGGCGAATTGGGTACCCTCGAACCCACCTCGGCCAC | This study |
| CTX-Kpn-IF-cas-R | CTCGAGGGGGGGCCCGGTACCCGTCGTGCATCTGGGACTCG | This study |
| CTX-Kpn-Cas9-F | CTATAGGGCGAATTGGGTACCATGGGCATAAAGTTGCCTCG | This study |
| CTX-Kpn-Cas9-R | CTCGAGGGGGGGCCCGGTACCCCATACCCATGGGTATTTACCAC | This study |
| CTX-SalI-pKD46-F | GGGCCCCCCCTCGAGGTGTATTTAGAAAAATAAACAAATAGGGGT | This study |
| | TCC | |
| CTX-SalI-pKD46-R | AAGCTTATCGATACCGTCGACCATGGATTCTTCGTCTGTTTCTACT | This study |
| | G | |
| cas1-F | ATTCGCACTACTGGAACATCC | This study |
| cas1-R | CCGCAGAAGCCTACCAATAC | This study |
| cas2-3-F | TTTCAGCCGCTACGAAGAAG | This study |
| cas2-3-R | GACTAGCTCCTCCCGATAGTT | This study |
| cas8-F | CCGCAGAAGCCTACCAATAC | This study |
| cas8-R | GGAGAACTGGTTGCTACCTTC | This study |
| cas5-F | GCTTCTCCCGAAGTCATGTT | This study |
| cas5-R | AATGCCCGTGATAGGGAAAC | This study |
| cas7-F | GATACCAAAGCAGGTCCGATAA | This study |
| cas7-R | AAGCAGAAGCTGGACTTCTATAC | This study |
| cas6-F | GATCAGGTTGGCGATCACATA | This study |
| cas6-R | CAGCACTTCCGTCTCTTCAT | This study |
| gam-F | GTTCAGCCATGAACGCTTATTAC | This study |
| gam-R | AGTTCTGCCTCTTTCTCTTCAC | This study |
| beta-F | GTTCAGCCATGAACGCTTATTAC | This study |
| beta-R | AGTTCTGCCTCTTTCTCTTCAC | This study |
| exo-F | GGTGGTTTCGAGGCCATAA | This study |
| exo-R | CTTCATACGCGGGTCATAGTT | This study |
| acf-F | GTCAGCAGTTCGACGATTCA | This study |
| acf-R | TAAGCCACTCGCCATCTTTC | This study |
| aca-F | TCGACAAAGCAGGTGTCAG | This study |
| aca-R | TGCGAACTGAACCGTGTAA | This study |
| rhII-F | CGTCGGTCTGGGAGCTTTCG | This study |
| rhII-R | CCCAGGTACCAGGCGCATTG | This study |

Construction of Transferable Cas System

The transferable cas system was derived from the mini-CTX-lacZ plasmid. The mini-CTX-lacZ plasmid was linearized under HindIII (NEB, USA) treatment for 4 hours. The Ptat promoter was amplified from the PA154197 genome by PCR using the iProof High-Fidelity DNA Polymerase (Bio-Rad, USA) and ligated with the HindIII-digested mini-CTX-lacZ plasmid using the ClonExpress II One Step Cloning Kit (Vazyme, China), generating CTX-Ptat-lacZ plasmid. The I-F cas operon including its native promoter was amplified from the PA154197 genome. Fragments encoding λ-red genes with the L-arabinose inducible promoter was amplified from the pKD46 plasmid. The cas operon and λ-red encoding fragment were inserted into the KpnI and SalI sites in the CTX-Ptat-lacZ plasmid, respectively, to generate the transferable I-F cas system and transferable λ-I-F cas system. For the construction of the transferable λ-Cas9 system, the cas9 gene and its promoter was amplified from the pCasPA.

Integration of the Transferable Cas System

E. coli SM10 strain containing the transferable cas system was cultured in LB broth supplemented with 10 μg/ml tetracycline at 37° C. with 220-rpm agitation for about 16 h. Simultaneously, P. aeruginosa recipient strain was cultured in LB broth at 42° C. with 220-rpm agitation for about 16 h. Cell densities of the E. coli SM10 and P. aeruginosa cultures were determined by measuring OD600 nm, then E. coli SM10 and P. aeruginosa were mixed with the cell number of 1.5×10⁹ and 0.5×10⁹. Mixture was pelleted by centrifugation at 16,000×g for 1 min and resuspended in 50 μl LB broth followed by spotted on the surface of a LB agar plate. Mating (plasmid delivery from SM10 to P. aeruginosa) occurred during the incubation of the mixture at 37° C. for 8 h. Mixture was scrapped from the agar plate and resuspended in 300 μl PBS buffer. Cell suspension was serial diluted and spread on the VBMM plates with supplementation of 50 μg/ml tetracycline and 40 μg/ml X-gal and plates were incubated at 37° C. for 24 h. Blue colonies indicating the chromosomal integration of the transferable cas system are selected for further use.

Quantitative PCR (qPCR)

Expression of the integrated genes such as cas and λ-red genes was detected by qPCR which was performed as described previously (Xu, et al. Journal of Biological Chemistry 294, 16978-16991, doi:10.1074/jbc.RA119.010023 (2019)). 1 ml bacterial cells grown in LB were harvest when the OD600 nm reached 1.0. Total RNA was extracted using the Takara MiniBEST Universal RNA Extraction Kit (Takara, Japan) and reverse transcription were conducted using the PrimeScript RT Master Mix (Takara, Japan) following the manufacturer's instructions. qPCR was performed using specific primers mixed with the TB Green Premix Ex Taq (Takara, Japan) in a 20 μl reaction system. The amplification was performed in the ABI StepOnePlus real-time PCR system. Amplification curves were plotted to display the transcription of tested genes and Ct value was used to compare the relative transcription levels in different strains. The recA gene was selected as the internal reference gene.

Construction of pTargeting and pEditing

A plasmid (pAY-mini-CRISPR) encompasses a 32-bp spacer insertion site flanked by two repeats (GTTCACTGCCGTATAGGCAGCTAAGAAA) is designed to assist the construction of specific mini-CRISPR elements. 32-bp nucleotides (spacer) preceded by a 5′-CC-3′ PAM is selected in the coding sequence of the gene to be edited. Two oligos of the spacer DNA are designed in the following form: 5′-GAAANx32-3′ and 5′-GAACNx32-3′. Oligos are first phosphorylated using T4 polynucleotide kinase (NEB, USA) at 37° C. for 1 h. The phosphorylated oligos are heated at 95° C. for 3 min and then cooled down to room temperature to generate annealed oligos. Annealed oligos are ligated into the plasmid pAY-mini-CRISPR which is pre-digested with BsaI (NEB, USA) using the Quick Ligation™ Kit (NEB, USA) to generate the desired mini-CRISPR. Assembly of mini-CRISPR and donor template into the platform plasmid (pAY5211) was followed our previously published protocol (Xu, & Yan, STAR Protocols 1, 100039, (2020)), the entirety of which is incorporated herein by reference. Specifically, amplified mini-CRISPR elements and plasmid pAY5211 were digested using KpnI and BamHI (NEB, USA) and ligated using the Quick Ligation™ Kit (NEB, USA) to generate the targeting plasmid (pTargeting). Donor sequences which contain upstream and downstream homologous arms of the gene being edited with 21-bp overlap of the XhoI-digested targeting plasmid at each end were amplified by PCR and ligated into the linearized targeting plasmid (digested by XhoI (NEB, USA)) using the ClonExpress II One Step Cloning Kit (Vazyme, China) to generate the editing plasmid (pEditing). All constructed plasmids were verified by Sanger sequencing (BGI, China).

Quantification of Conjugation Efficiency

E. coli SM10 strains containing the plasmids of pAY5211 and pAY7138 were cultured in LB broth supplemented with 100 μg/ml kanamycin at 37° C. with 220-rpm agitation for about 16 h while the P. aeruginosa strains (tetracycline resistance due to the integrated transferable cas system) were cultured in LB broth at 42° C. with 220-rpm agitation for about 16 h. E. coli SM10 and P. aeruginosa were mixed with the cell number of 1.5×10⁹ and 0.5×10⁹. Mixture was pelleted by centrifugation at 16,000×g for 1 min and resuspended in 50 μl LB broth followed by spotted on the surface of a LB agar plate. The plate was incubated at 37° C. for 8 h. Mixture was scrapped from the agar plate and resuspended in 300 μl PBS buffer. Cell density was determined by measuring OD600 nm and adjusted to 2.0. Cell resuspensions were serially diluted and plated on the LB agar plates containing 50 μg/ml tetracycline and 500 μg/ml kanamycin. Plates were incubated at 37° C. for 24-36 h. Original amounts of survival cells in the resuspension was determined by recovered colony numbers and dilution factors. Colony number recovered from pAY5211 was normalized as 100%.

Genome Editing and Verification

E. coli SM10 strain containing the editing plasmid was cultured in LB broth supplemented with 100 μg/ml kanamycin at 37° C. with 220-rpm agitation for about 16 h while the P. aeruginosa strains (tetracycline resistance due to the integrated transferable cas system) were cultured in LB broth supplemented with 20 mM L-arabinose at 42° C. with 220-rpm agitation for about 16 h. E. coli SM10 and P. aeruginosa were mixed with the cell number of 1.5×10⁹ and 0.5×10⁹. Mixture was pelleted by centrifugation at 16,000×g for 1 min and resuspended in 50 μl LB broth followed by spotted on the surface of a LB agar plate containing 20 mM L-arabinose. The plate was incubated at 37° C. for 8 h. Mixture was scrapped from the agar plate and resuspended in 300 μl PBS buffer. For most strains expect kanamycin resistant strains, cell resuspension was spread on the LB agar plates containing 50 μg/ml tetracycline and 500 μg/ml kanamycin. Plates were incubated at 37° C. for 24-36 h. Recovered single colonies were inoculated into LB broth containing 100 μg/ml kanamycin in a 96-well plate. After incubation at 37° C. with 220-rpm agitation for 3 h, luminescence intensity was measured and ten colonies with the highest luminescence intensity were subjected to verification using PCR and Sanger sequencing with specific primers. PCR results of ten selected clones in each experiment were presented to show the efficiency of genome editing. The editing plasmid in the P. aeruginosa cells which underwent one round of editing is cured by streaking the cells onto a LB ager plate and incubation at 37° C. overnight. In some cases, multiple (2 to 3) rounds of streaking are required for the thorough plasmid curing.

Bacterial Whole Genome Sequencing and Analysis

Genomic DNA was extracted from 1 ml overnight bacterial culture using the Illustra bacteria genomic Prep Mini Spin Kit (GE Healthcare, USA) according to the manufacturer's instructions. Whole genome sequencing was conducted by Novogene (Beijing, China) with the sequencing platform Novaseq. The quality control of raw reads was done using Trimmomatic. The genome of the PAO1^{λIF} strain was assembled using SPAdes with PAO1 (NC_002516.2) as the reference, and completed using an assembly improvement pipeline. Circlators fixstart task was used to fix the start position of the manually finished complete genome of PAO1^{λIF} to be at the dnaA gene. Annotations of PAO1^{λIF} were done using Prokka with Pseudomonas genera specific database. With the complete genome of PAO1^{λIF} as the reference, the genome-wide integration site distribution for a given strain was done referring to one previous study. Mapping in the present study was performed using BWA. Integration events within 500 bp bins were computed using SAMtools and BEDTools47 and visualized with ggplot2 in R platform (https://ggplot2.tidyverse.org/index.html). To explore mutations on all other strains compared with PAO1^{λIF}, core SNPs were collected and annotated with the reference of the PAO1^{λIF} genome using snippy (https://github.com/tseemann/snippy).

PYO Quantification

1 ml bacterial culture was subjected to centrifugation at 16,000×g for 5 min. 750 μl supernatant was collected and mixed with 450 μl chloroform by vortex for 0.5 min. After centrifugation at 16,000×g for 5 min, 400 μl liquid from lower phase was then mixed with 200 μl HCl (0.2 M) by vortex thoroughly for 0.5 min. After centrifugation at 16,000×g for 5 min, 100 μl upper aqueous phase containing PYO was transferred to a 96-well plate and its absorbance was measured at 510 nm. Concentration of PYO was determined according to the standard curve.

### Results

Previous studies identified a highly active I-F CRISPR-Cas system from P. aeruginosa PA154197 which encompasses six cas genes sandwiched by two convergent CRISPR arrays (FIG. 1A). This system recognizes a canonical 32-bp protospacer preceded by a di-nucleotide protospacer adjacent motif (PAM) of 5′-CC-3′. Five Cas proteins, Cas8f (Csy1), Cas5 (Csy2), Cas7 (Csy3), and Cas6f (Csy4) which are assembled in the Cascade and Cas2-3 which contains helicase and nuclease domains, are involved in the target DNA recognition and cleavage under the guidance of a crRNA. To harness this system for generic genome editing, a chromosomal integration-mediated transferable I-F cas system was designed instead of employing plasmid-encoded Cas proteins which suffer from the limitation of narrow host range and requires antibiotic to maintain its propagation and expression (FIG. 1B). Given that the poor capacity of intrinsic homologous recombination in most bacterial hosts, a phage λ-red recombination system is further assembled for the homology directed repair (HDR)-mediated genome editing, generating another transferable λ-I-F cas system (FIG. 1C). When the transferable systems are introduced into P. aeruginosa strains, they can integrate efficiently into the specific attB site, which is highly conserved among the P. aeruginosa species. A lacZ reporter driven by a strong promoter Ptat is designed in the transferable systems so that chromosomal integration of the system can be easily detected on the 5-bromo-4-chloro-3-indolyl-β-D-galactopyranoside (X-gal)-containing plates, which simplifies the verification regardless of the un-conserved sequences flanking the attB sites in distinct clinical strains. When the recipient P. aeruginosa strain acquired the ‘native’ type I-F cas system, its activity is readily examined by introducing a targeting plasmid pTargeting and desired genome editing is achieved in one step by introducing an editing plasmid pEditing, respectively (FIG. 1D and FIG. 1E). After editing, the entire integrated element is removed by another round of editing using a CRISPR-Cas removal plasmid.

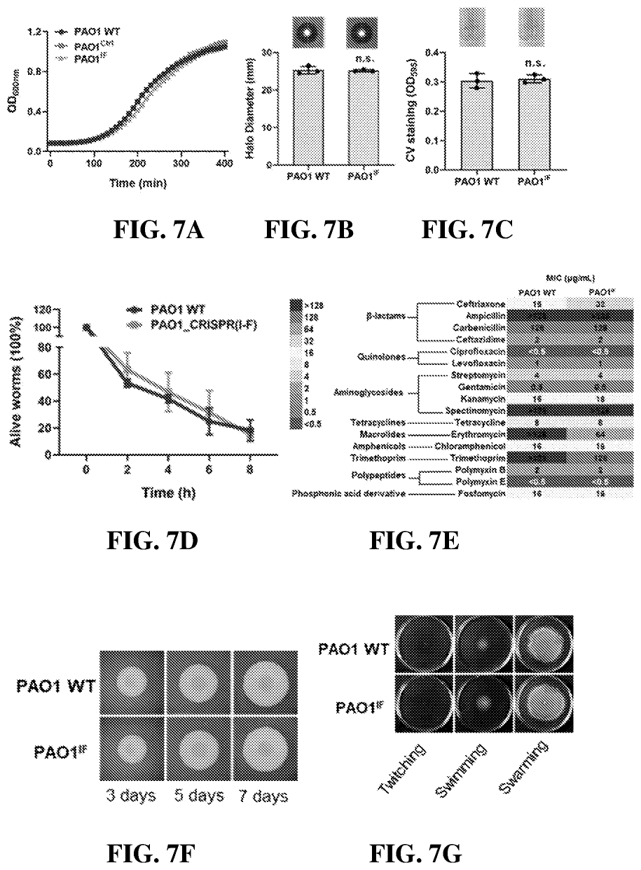

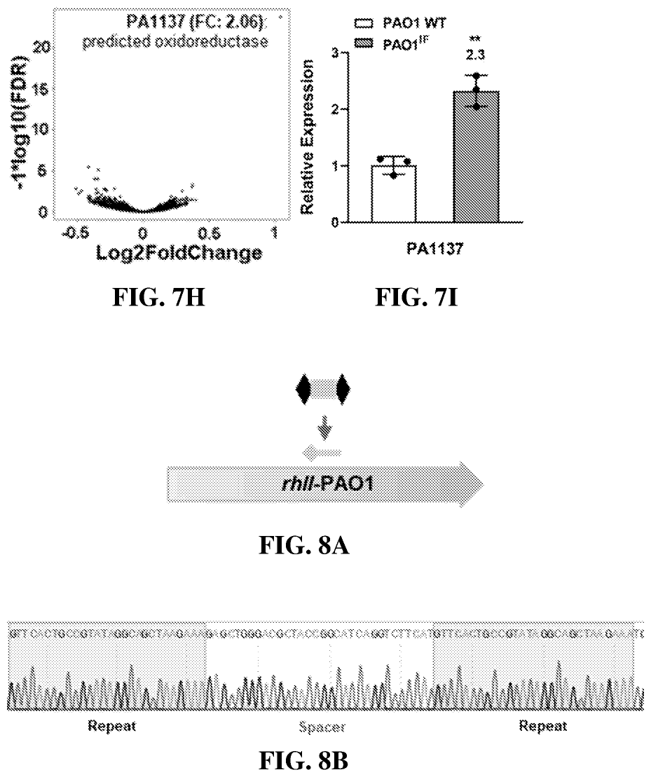

When the transferable I-F and λ-I-F cas systems were constructed, the CRISPR-free model strain PAO1 was first selected to examine their integration efficiency as well as the expression and interference activities. All the recovered clones appeared in blue by introducing the transferable systems into PAO1 (FIG. 1F), suggesting the successful chromosomal integration and expression of the lacZ gene in all the recovered cells. One blue clone integrated with the λ-I-F cas system was randomly selected and subjected to whole genome sequencing (WGS), which revealed that the integration is site-specific at the attB site (Genomic location: 2,947,580-2,947,610) (FIG. 1G) and only a 4-bp synonymous substitution was identified (Table 2). This result indicated the highly efficient and precise integration of the vectors. Expression of all the cas genes was only detected in PAO1^{IF} rather than PAO1^{Ctrl} which is a control strain integrated with a transferable system lacking the cas operon (FIG. 1H). To ensure the chromosomal integration will not affect bacterial physiology, a series of assays were performed to systematically examine the potential consequences of this integration. No difference on cell growth, proteolytic activity, biofilm formation, C. elegans killing, antibiotic susceptibility, colony morphology and motility was observed between the PAO1^{IF} and PAO1 WT strains (FIGS. 7A-7G). The two strains were further compared at the transcriptome level and only one gene PA1137 encoding a predicted oxidoreductase was detected with 2-fold upregulation in PAO1^{IF} (FIGS. 7H-7I). These results demonstrated that integration of the transferable system yields no significant effect on bacterial physiology, enabling the exploration of following genome editing.

TABLE 2
WGS revealed mutations between the
indicated strains and PAO1^{λIF}

| Strain | Position | Strand | Gene | Effect |
|---|---|---|---|---|
| PAO1 WT | 2015344 | − | ccoN1_3 | synonymous_variant: |
| | | | | CCAC::ACAT at |
| | | | | 738 to 741 |
| False | 2954847 | + | cas2-3 | frameshift_variant: |
| positive | | | | deletion from |
| No. 1 | | | | 1349 to1380 |
| False | 2958217 | + | cas5 | stop_gained: C139T |
| positive | | | | |
| No. 2 | | | | |
| False | 2958217 | + | cas5 | stop_gained: C139T |
| positive | | | | |
| No. 3 | | | | |

The interference activity of the transferred CRISPR-Cas system using self-targeting assay was tested. A targeting plasmid pRhlI-Targeting was constructed based on the platform plasmid pPlatform (pAY5211) that was developed. The mini-CRISPR in pRhlI-Targeting encompasses a 32-bp spacer that is complementary to a “5′-CC-3′″-preceded protospacer within the rhlI gene in PAO1 (FIGS. 8A-8B). Introduction of pRhlI-Targeting led to a significantly decreased (>10⁻⁵) efficiency of conjugation relative to the control plasmid pAY5211 in PAO1^{IF} but not in PAO1^{Ctrl} (FIG. 1I), indicating the transferred I-F cas system in PAO1 is highly active to execute genome cleavage. To test the applicability of the transferable cas system in different hosts, the system was integrated to another model strain PA14 and 30 randomly selected clinical isolates from Queen Mary Hospital (Hong Kong). It was shown that the system can be integrated into all the strains and cas genes therein can express ordinary (FIG. 9A). Given that the rhlI genes are not identical in all the strains which may lead to the escape of self-targeting, a universal targeting plasmid pAY7138 which encodes a crRNA targeting to the Ptat promoter located upstream of the lacZ gene in the transferable system was designed (FIGS. 9B and 9C). Interference activities of the transferred CRISPR-Cas systems in all the recipient strains were examined by comparing the relative conjugative efficiency between pAY5211 and pAY7138. As shown in the FIG. 9D, 10 out of 30 strains showed active interference in the presence of the transferred CRISPR-Cas system, representing a high percentage of recipient host range.

### Example 2: Comparison of the Transferable Type I and Type II CRISPR-Cas Systems

### Results

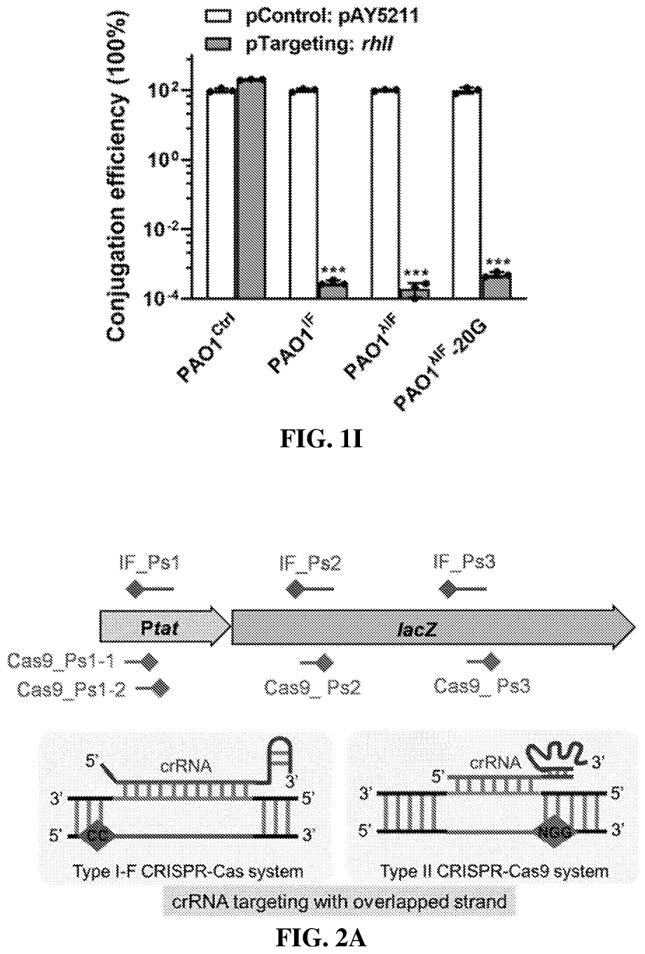

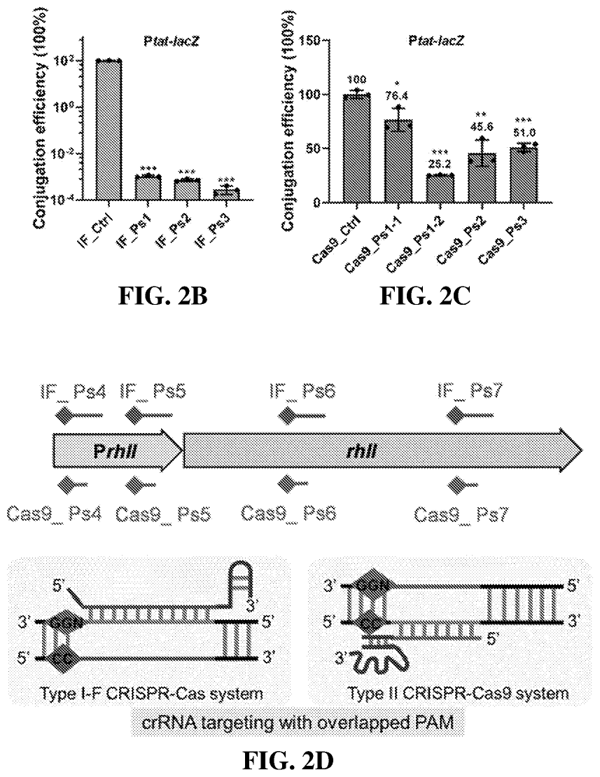

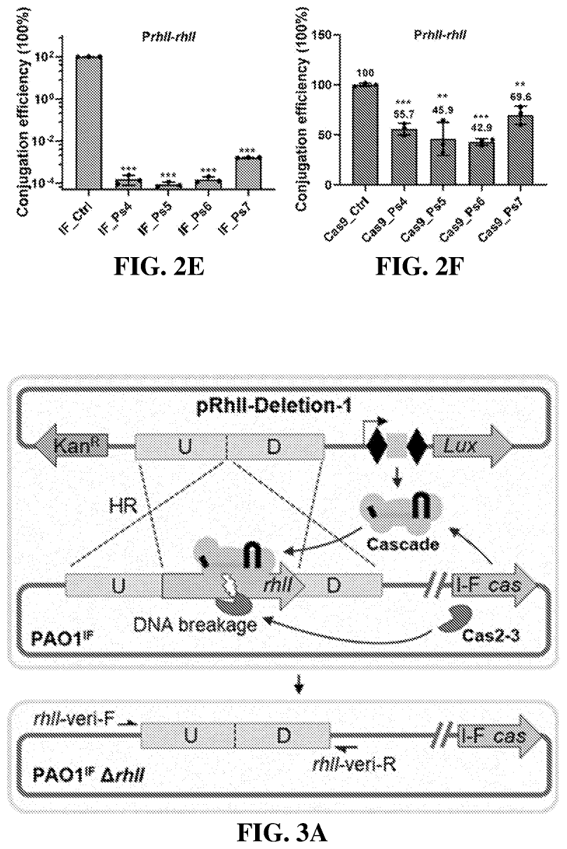

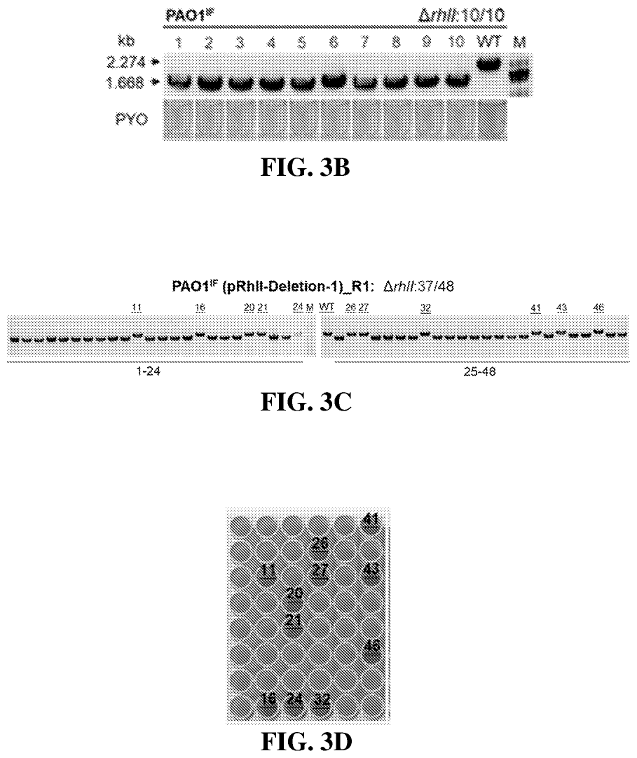

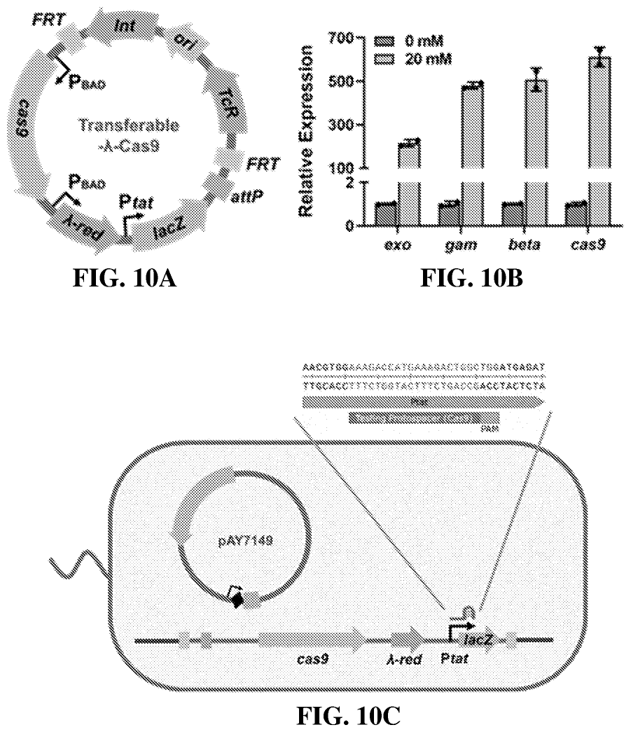

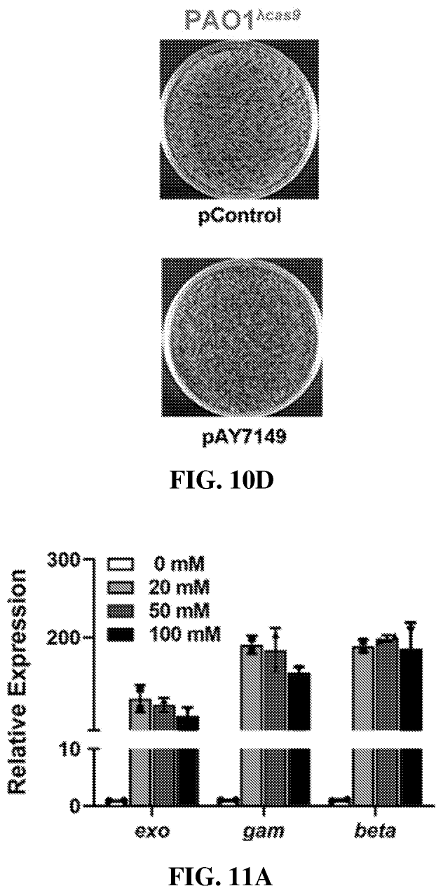

It was reported that transcription activator fused to the type I-F Cascade has a higher activation level compared with its fusion to the type II dCas9 protein in human cells. The targeting efficiency of the transferable type I-F and type II CRISPR-Cas systems in bacterial cells was compared. A transferable λ-Cas9 system was constructed by replacing the I-F cas operon in the transferable λ-I-F cas system with the Spcas9 gene and confirmed the expression of cas9 and λ-red genes using RT-qPCR after the system was integrated into the PAO1 genome (PAO1^{Cas9}) (FIGS. 10A-10B). To test the targeting efficiency of the transferred Cas9 system, a universal targeting plasmid pAY7149 which encodes a crRNA targeting a protospacer Cas9_Ps1-1 was designed. Cas9_Ps1-1 is partially overlapped with the protospacer IF_Ps1 which is recognized by the pAY7138-encoded crRNA (FIG. 2A and FIG. 10C). Interestingly, introduction of pAY7149 into PAO1^{Cas9} did not lead to reduced transformants compared to the control plasmid (FIG. 10D), which only caused 23.6% reduction of conjugation efficiency (FIG. 2C). In contrast, Conjugation efficiency was significantly reduced (10⁻⁵) by introducing pAY7138 into PAO1^{λIF} compared to the control plasmid (FIG. 2B). To exclude the possibility that the selected protospacer is an ineffective one for Cas9-based targeting, additional protospacers in the promoter Ptat (Cas9_Ps1-2) and the lacZ gene (Cas9_Ps2 and Cas9_Ps3) were tested for self-targeting examination (FIG. 2A). For comparison, corresponding protospacers (IF_Ps1 to 3) that overlap the Cas9 protospacers were selected to quantify the self-targeting effect mediated by the I-F CRISPR-Cas system. As the result shown in FIG. 2C, despite targeting effects of the Cas9 system exist at different genomic locus, their efficiencies are greatly lower than the I-F system. Four more protospacers located in the endogenous rhlI gene and its promoter region were examined. I-F and Cas9 systems share the same PAM sequences at these four sites but the crRNAs for two systems target two different strands (FIG. 2D). Similarly, I-F systems exhibited higher targeting efficiency than the Cas9 system (FIGS. 2E and 2F). The transferable I-F CRISPR-Cas system was selected for genetic manipulations owing to its higher targeting efficiency.

### Example 3: Transferable I-F Cas Systems Enable Diverse Genetic Manipulations in PAO1

### Results

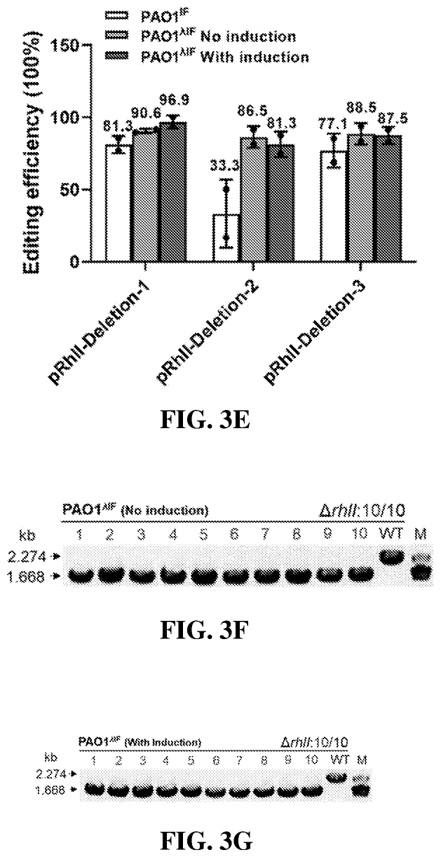

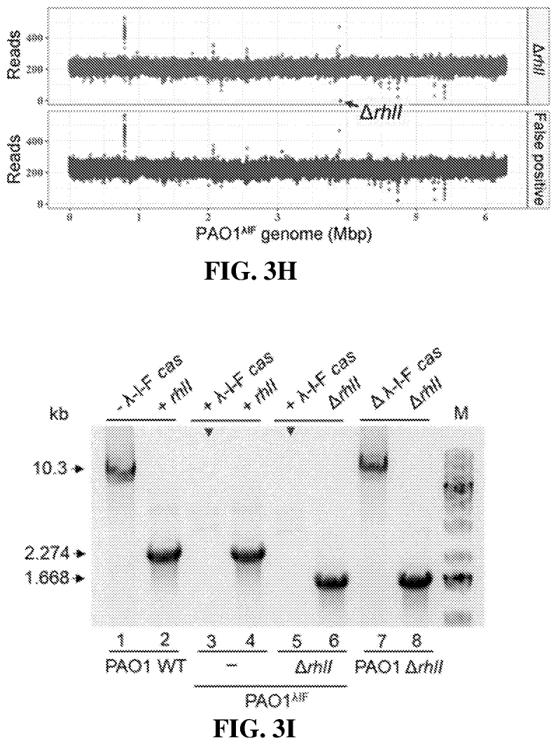

As the targeting activity of the transferable I-F CRISPR-Cas system was confirmed, this system was then exploited for gene deletion. The rhlI gene as selected for deletion because mutants with dysfunctional Rhl system in P. aeruginosa showing an obvious phenotype change due to its inability to produce the blue pigmented pyocyanin (PYO). A rhlI-deleting plasmid (pRhlI-Deletion-1) was constructed by assembling ˜800-bp upstream and ˜800-bp downstream homologous arms of rhlI into the targeting plasmid (pRhlI-Targeting) for homologous recombination (FIG. 3A). Introducing pRhlI-Deletion-1 into PAO1^{IF}, 48 randomly selected clones were inoculated and subjected to luminescence screening. The genotypes of 10 clones showing the highest luminescence signal were further analyzed by colony PCR, which confirmed that all of them harbored the desired rhlI deletion (FIG. 3B). This result demonstrated the first success of employing transferable type I cas system for efficient genome editing in the heterologous bacterial host. To further evaluate the editing efficiency with a larger population of recovered clones, we first determined whether abolished PYO production can be used to indicate the rhlI deletion. We verified the genotypes of all the 48 selected clones by colony PCR and identified 37 out of them were the desired rhlI-deleted mutants (FIG. 3C).

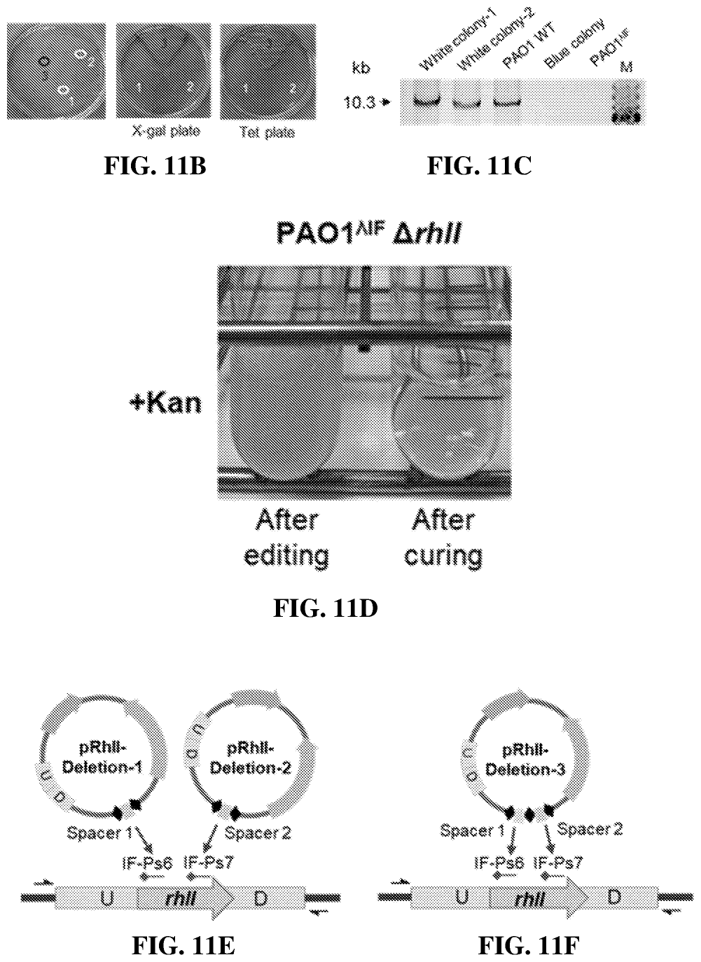

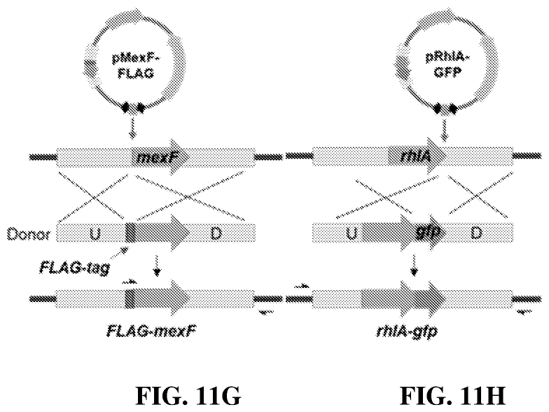

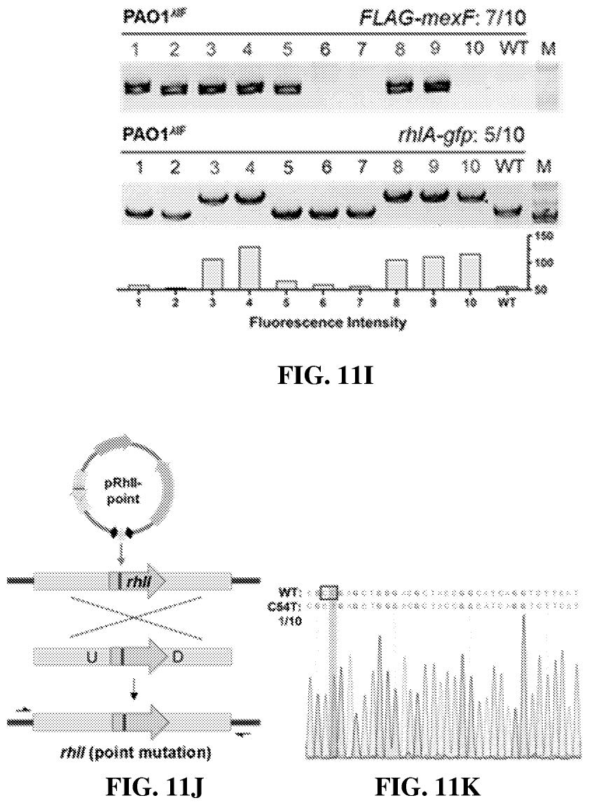

Consistent with the PCR result, abolished PYO biogenesis was observed in the corresponding 37 identified rhlI mutants (FIG. 3D), indicating that disrupted PYO biogenesis can be used to facilitate the selection of rhlI-deleted mutants. From two independently repeated assays by introducing pRhlI-Deletion-1 into PAO1, 81.3% efficiency of rhlI deletion was obtained in average (FIG. 3E). To Investigate if the presence of the λ-red recombinase could further elevate the editing efficiency, we performed rhlI deletion in PAO1^{MI}F. We introduced the editing plasmid pRhlI-Deletion-1 into PAO1^{λIF} with or without treatment of 20 mM L-arabinose which was found sufficient to induce the highest expression levels of the λ-red genes (FIG. 11A). Facilitated by luminescence screening, 10/10 clones with the highest luminescence signal were found harboring the desired rhlI deletion in the both absence and presence of L-arabinose induction (FIGS. 3F and 3G).

We next asked the specificity of the transferable I-F cas-mediated genome editing and the potential causes of false positive clones. 3 PCR-confirmed rhlI-deleted clones (ΔrhlI) and 3 false positive clones generated from PAO1^{λIF} were selected for WGS analysis. Precise rhlI deletion without additional off-target mutation was identified in ΔrhlI (FIG. 3H), demonstrating the type I-F cas-mediated genome editing is highly site-specific. Interestingly, mutations in the cas genes were identified in all the three false positive clones, i.e. 33-bp deletion in cas2-3 and point mutations resulting in the premature of cas5 (Table 2), which directly inactivated the Cas system and caused the failure of self-targeting.

To obtain the PAO1 mutant that only contains the desired rhlI deletion (PAO1 ΔrhlI) without the redundant 21.212-kb transferred elements, the capacity of the transferred λ-I-F cas system was further explored to delete large-scale genomic fragments such as the transferred elements. However, deletion of the entire integrated sequence would be extremely difficult owing to its considerably large size. To achieve this, the lacZ reporter was employed therein to indicate the excision same as the integration process. Once the integrated sequence which contains the constitutively expressed lacZ gene is removed, the recovered cell is unable to digest the X-gal substrate and consequently the colony grows in white color. A CRISPR-Cas removal plasmid pAY7401 which encompasses a lacZ-targeting mini-CRISPR and a donor sequence consisting of 5,067-bp upstream and 5,082-bp downstream homologous arms of the attB site was constructed. The excision capacity was first tested in the PAO1^{λIF} strain by introducing pAY7401 into this strain and recovered them on X-gal containing plates. As shown in the FIG. 11B, white clones were recovered, suggesting the excision of the integrated lacZ-containing transferable cas system. Given that the integrated system contains a tetracycline-resistant gene, the excision was also verified by the susceptibility of the white clones against tetracycline. Abolished growth of the selected white clones in the presence of tetracycline indicated the loss of the tetracycline-resistant gene (FIG. 11B). By amplifying the 10.3-kb region flanking the attB site (primers are located outside the donor sequences), it was demonstrated that the integrated transferable λ-I-F cas systems were removed in the two white clones because PCR products from these two clones showed the same size as the PAO1 WT (FIG. 11C). (Note that no PCR band in the lanes of blue clone and PAO1^{λIF} in FIG. 11C is because our PCR polymerase was unable to amplify the target sequence in these strains owing to the extremely large size (21.212-kb integrated sequence and its 10.3-kb flanking sequences)). Next, to generate the PAO1 ΔrhlI strain, the transferable λ-I-F cas system was removed by introducing pAY7401 in PAO1^{λIF} ΔrhlI after the curing of pRhlI-Deletion in this strain (FIG. 11D). Deletion of the rhlI gene and excision of the transferable system in PAO1 ΔrhlI were verified by PCR, which showed 600-bp reduction of rhlI and the same size of product flanking the attB site as the PAO1 WT (FIG. 3I). Together, these results demonstrated that the transferable λ-I-F cas system is capable of deleting large-scale genomic fragments and the system can be removed with convenient selection after desired genome editing is achieved.

Furthermore, the effect of spacer numbers in editing efficiency was compared. Two more rhlI-deleting plasmids pRhlI-Deletion-2 and pRhlI-Deletion-3 were generated (FIGS. 11E and 11F). pRhlI-Deletion-2 encodes a crRNA targeting the protospacer IF_Ps7 and pRhlI-Deletion-3 encodes two crRNAs targeting both IF_Ps6 and IF_Ps7. Screened by PYO production, 33.3% and 77.1% of PAO1^{IF} clones which were recovered from the introduction of pRhlI-Deletion-2 and pRhlI-Deletion-3, respectively, carried the expected rhlI deletion (FIG. 3C). pRhlI-Deletion-3 showed comparable editing efficiency with pRhlI-Deletion-1 and the relatively low editing efficiency of pRhlI-Deletion-2 was possibly due to the lower targeting efficiency at IF_Ps7 compared to IF_Ps6 (FIG. 2E). These results suggested that editing efficiency varied with protospacers at different loci and targeting multiple protospacers could potentially prevent the inefficient targeting albeit employing pRhlI-Deletion-3 did not show improvement of editing compared with pRhlI-Deletion-1 (FIG. 3C). Moreover, other types of precise editing based on the transferable system such as gene insertion and point mutation were demonstrated (FIGS. 11G, 11H and 11J). As shown in FIG. 11I, N-terminal FLAG-tagging in mexF and C-terminal gfp-tagging in rhlA were achieved with comparable efficiency of rhlI deletion. Regarding to the point mutation (C54T) we explored in rhlI, although the editing efficiency is relatively low (1/10), desired mutation can still be obtained in one-step of transformation (FIG. 11K).

### Example 4: Transferable λ-I-F Cas System Enables Genome Editing in Strains with Various Genetic Background

### Results

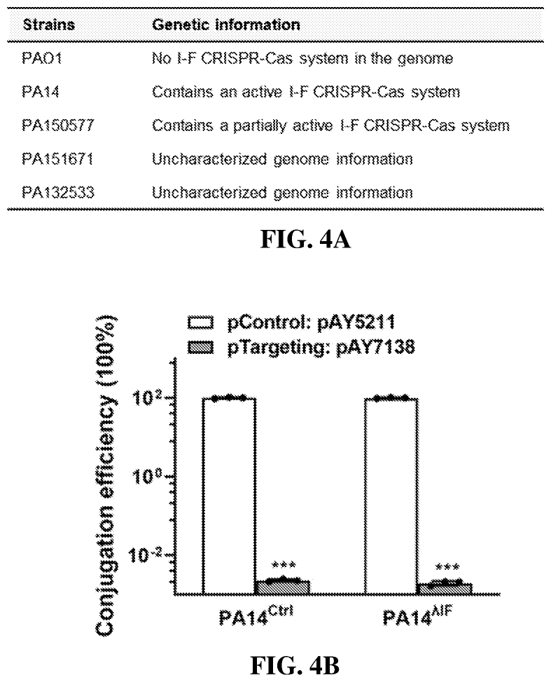

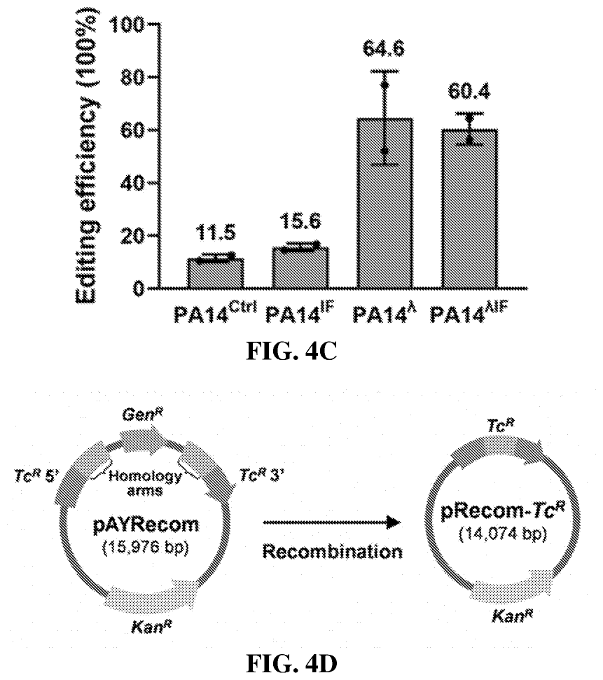

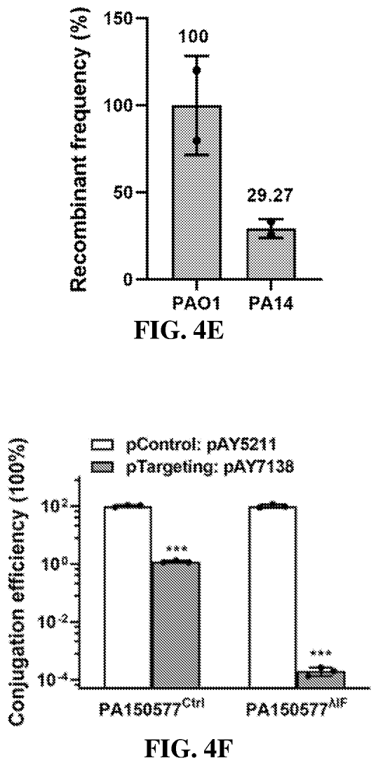

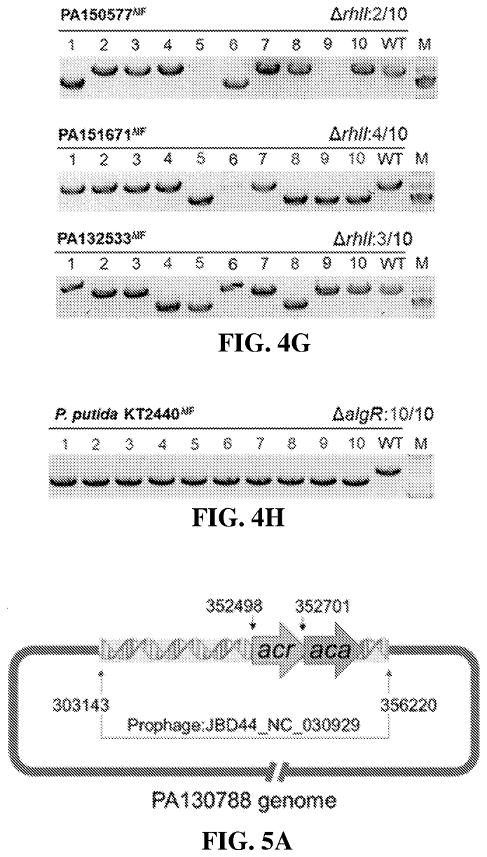

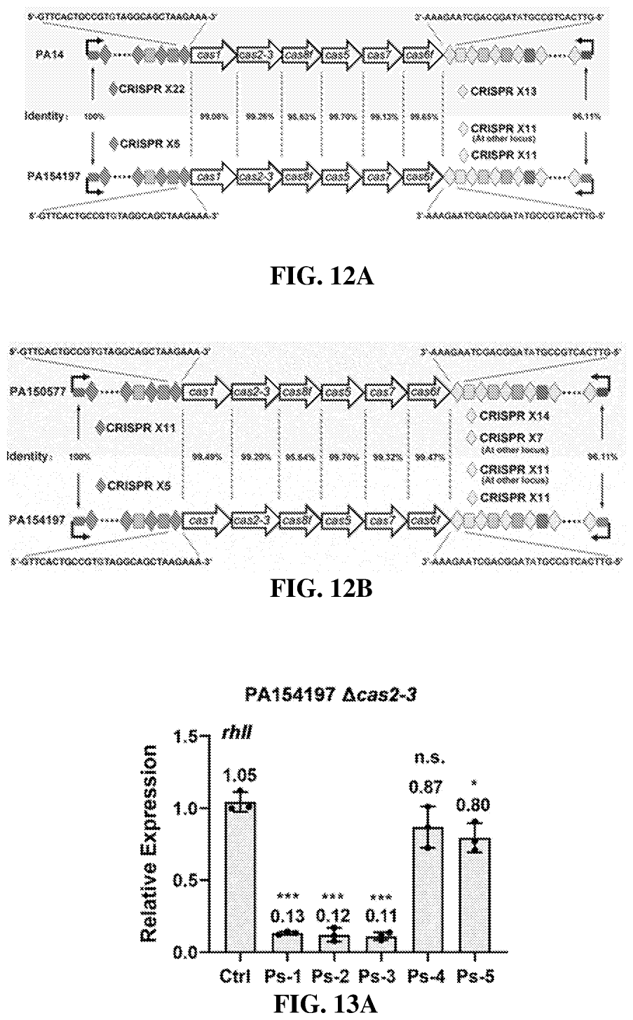

This transferable λ-I-F cas system for genome editing was expanded in other strains with different genetic background. In addition to the CRISPR-free strain PAO1, we selected some representative I-F CRISPR-Cas-containing strains but with different activities as well as some un-sequenced strains (FIG. 4A). PA14 contains a fully active I-F CRISPR-Cas system (FIG. 4B), PA150577 contains a I-F CRISPR-Cas system with compromised activity (FIG. 4F), PA151671 and PA132533 are un-sequenced isolates without genome information. In PA14, its native I-F CRISPR-Cas system is active to conduct self-targeting when pAY7138 was introduced into PA14_Control (FIG. 4B), which suggested the potential of harnessing its native system for gene deletion. When pRhlI-Deletion-3 was introduced into the cell, rhlI deletion was achieved but with relatively low efficiency (11.5% based on PYO selection) (FIG. 4C). Comparable efficiency (15.6%) was obtained in PA14 with the transferred I-F cas system (FIG. 4C), suggesting that the low editing efficiency in PA14 was not due to the different host origins of the cas systems. In fact, type I-F CRISPR-Cas systems from PA14 and PA154197 shared 98.75% identity in sequence (FIG. 12A). Thus, low editing efficiency in PA14 was speculated as the result of its poor intrinsic recombination capacity. To test this speculation, an assay was performed to quantify the recombination frequencies in PAO1 and PA14 (FIG. 4D). The result showed that the intrinsic recombination frequency in PA14 is only 29% relative to that of PAO1 (FIG. 4E), demonstrating the poor intrinsic homologous recombination capacity in PA14. Thus, to increase the editing efficiency in PA14, the λ-red recombination system was transferred into PA14 and examined its efficiency of rhlI deletion. As shown in FIG. 4C, efficiency of rhlI deletion was substantially increased to 64.6%. Similarly, integration of the transferable λ-I-F cas system in PA14 could also increase the editing efficiency to 60.4% (FIG. 4C). Combined, these results demonstrated that our transferable λ-I-F cas system not only mediated site-specific cleavage of target DNA but also increased recombination capacity for genome editing. Thus, the transferable λ-I-F cas system is preferred for genome editing in clinical strains. Additionally, these results indicated that the transferred cas system is compatible with the native system.

PA150577 is a clinically isolated strain which was found harboring a I-F CRISPR-Cas system in its genome. Sequence alignment showed that this system has a 98.69% identity with the I-F CRISPR-Cas system in PA154197 (FIG. 12B). This system is active to execute self-targeting when pAY7138 was introduced into PA150577^{Ctrl}. However, conjugation efficiency was decreased moderately which means that the activity of this system was partially repressed due to unknown reasons (FIG. 4F). This result suggested the infeasibility of harnessing its native CRISPR-Cas system for genome editing in PA150577. Interestingly, after PA150577 was transferred with our λ-I-F cas system, strong self-targeting was observed by introducing pAY7138 into the cells (FIG. 4F). This result indicated that the transferred λ-I-F cas system is active without inhibition by unknown repressors in PA150577 although this system is highly conserved with the one in PA150577 including the repeat sequences, spacer length, cas genes and their promoters (FIG. 12B). With the help of the transferred CRISPR-Cas system, deletion of rhlI was achieved (FIG. 4G). This interesting result further suggested that the native and transferred CRISPR-Cas systems may assemble independently without cross-interference. Further dissection on the sequence mutations in the cas genes may help to answer the discrepant interference activities of these two highly conserved systems. In addition, two strains PA151671 and PA132533 that were not sequenced but showed active self-targeting after integration of the transferable λ-I-F cas system were selected to explore genome editing (FIG. 9D). Since Rhl system is highly conserved in a majority of clinical strains, pRhlI-Deletion-3 were directly applied and rhlI deletion in both strains were successfully achieved with efficiency of 4/10 and 3/10, respectively (FIG. 4G). The applicability of the transferrable λ-I-F system was further examined in other Pseudomonas species, such as P. putida which is endowed with many traits desired for bioproduction and bioremediation. The λ-I-F cas system was readily integrated into the P. putida KT2440 genome. We then conducted editing using deleting the algR gene as an example in the resulting cells by one-step introduction of the editing plasmid pAYKT2440 via conjugation. A 10/10 (100%) editing efficiency was obtained (FIG. 4H). Together, all the results demonstrated that the transferable λ-I-F cas system enables genome editing in different strains with various genetic background including uncharacterized clinical isolates and other Pseudomonas species such as P. putida.

### Example 5: Presence of Anti-CRISPR Elements Restricts Genome-Editing

### Results

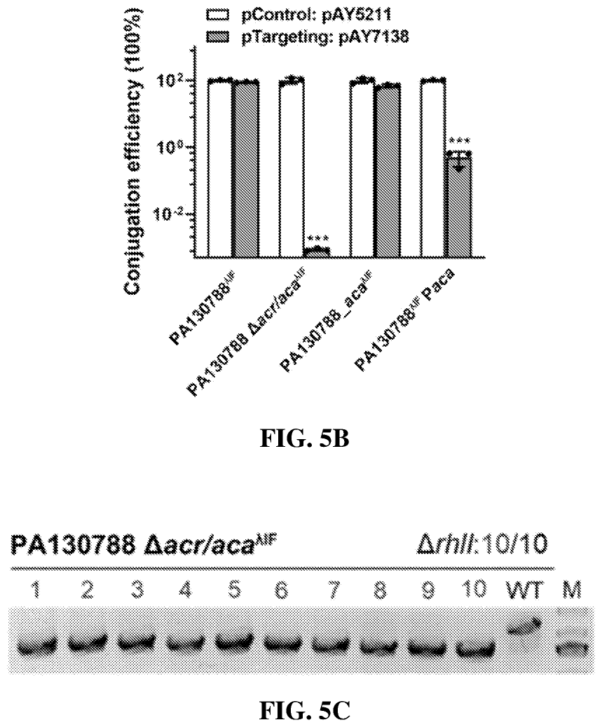

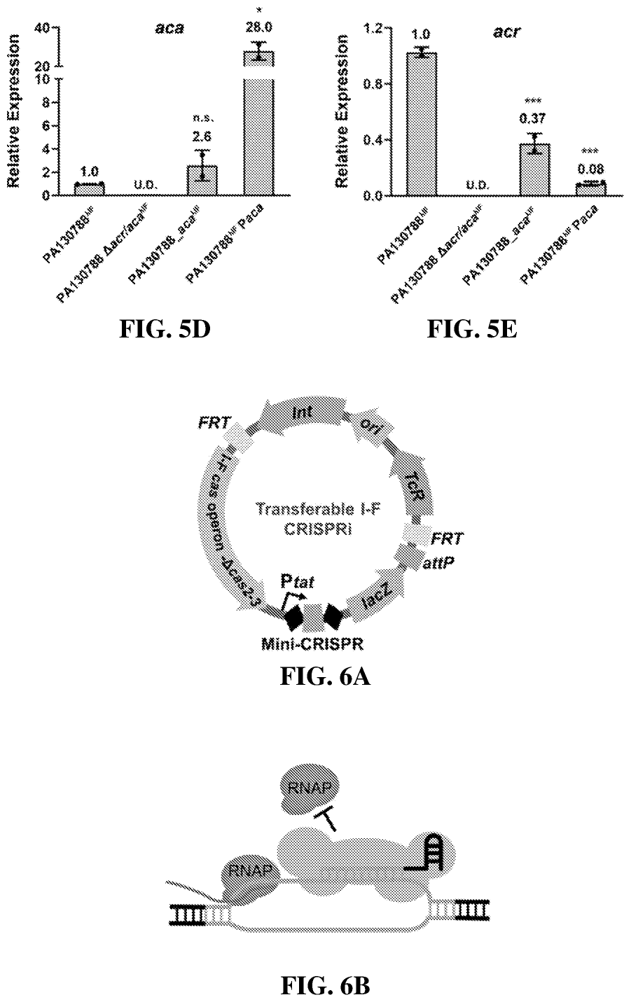

Although the transferable λ-I-F cas system provides a novel genome-editing strategy in P. aeruginosa, its activity is still inhibited in 2/3 screened clinical strains. The factors that inactivate the transferred CRISPR-Cas system were analyzed. Anti-CRISPR (Acr) is a group of natural inhibitors of CRISPR-Cas immune systems (Peng, et al., Trends in Microbiology, doi:10.1016/j.tim.2020.05.007; Marino, et al., Nature Methods 17, 471-479, doi:10.1038/s41592-020-0771-6 (2020)). So, the presence of anti-CRISPR elements could greatly prevent the editing process. Unfortunately, more than 30% of sequenced P. aeruginosa genomes were found to carry one or more Acr encoding genes (van Belkum, A. et al. Mbio 6, doi:10.1128/mBio.01796-15 (2015)). To confirm this obstacle, we sequenced the genome of PA130788, one of the strains with ineffective self-targeting, and searched for the presence of anti-CRISPR genes using AcrFinder (Yi, et al. Nucleic Acids Research 48, W358-W365, doi:10.1093/nar/gkaa351 (2020)). As speculated, an anti-CRISPR gene acr was identified (FIG. 5A). To verify and eradicate its inhibition role for genome editing, we next removed this gene together with its associated gene aca from the genome of PA130788 using the counterselection-based method (Choi & Schweizer, BMC Microbiol 5, 30-30, doi:10.1186/1471-2180-5-30 (2005)), generating a mutant PA130788 Δacr/aca. As shown in FIG. 5B, the transferred λ-I-F cas system in this mutant was reactivated to efficiently execute self-targeting, confirming that the failure of self-targeting in the PA130788^{λIF} strain was caused by the presence of anti-CRISPR element. When the anti-CRISPR gene was removed, 100% rhlI deletion was achieved (FIG. 5C).

Anti-CRISPR associated (aca) gene is commonly found located downstream of the acr gene and its function was recently proved to repress the expression of the acr genes. Based on that, a repression-based ‘anti-anti-CRISPR’ strategy was proposed to reactivate a CRISPR-Cas system that is inhibited by the presence of anti-CRISPR proteins and restore its potential for genome editing. Inspired by these studies, we sought to overexpress the aca gene in the PA130788^{λIF} strain to see if overproduced Aca proteins can repress the anti-CRISPR expression and restore the editing potentials. To stably and constitutively express aca without introducing additional plasmids, we first assembled the aca gene downstream of the Ptat promoter in the transferable λ-I-F cas system and integrated this system to PA130788, generating PA130788_aca^{λIF}. As a result, PA130788_ac^{λIF} displayed 2.6-fold upregulation of the aca gene and 2.7-fold downregulation of the acr gene compared to PA130788^{λIF} (FIGS. 5D and 5E), confirming the repression role of aca in the acr transcription. However, self-targeting assay showed neglectable reduction of conjugation efficiency in this strain (FIG. 5B), suggesting that the repressed acr was still sufficient to inactivate the CRISPR-Cas system. We then delivered an aca over-expressing plasmid Paca into PA130788^{λIF} and expected to further increase aca expression by its higher copy number than the chromosomal aca. This time, 28-fold upregulation of aca and 12.5-fold downregulation of acr were observed (FIGS. 5D and 5E). Although the self-targeting effect was significantly increased in the presence of plasmid-overexpressed Aca proteins, its level is still much weaker compared to the aca/acr deleted strain (FIG. 5B). Thus, repression of acr by the plasmid-overexpressed Aca is still not efficient for the next step of genome editing.

### Example 6: Repurposing the Transferable I-F CRISPR-Cas System for Gene Repression

### Results

In addition to the anti-CRISPR elements that inactivate the transferable CRISPR-Cas system, two other major factors that impede genome editing are the capacity of homologous recombination and the availability of the editing plasmid. Sometimes, it is difficult to obtain a replicating plasmid that is compatible to the strain of interest and the antibiotic-resistant capacity of a strain limits the use of antibiotic selection markers. For example, P. aeruginosa ATCC27853 (PA27853) exhibits intrinsic resistance to kanamycin which resulted in the incapability of our system for genome editing. To overcome such impediment, a transferable CRISPR-based transcriptional interference (transferable CRISPRi) system which allows alternative functional delineation of a gene without introduction of additional plasmid except the first integrative plasmid was developed. The CRISPRi system was devised by eliminating the cas2-3 gene in the transferable I-F cas system to disable its DNA cleavage ability (FIG. 6A). Upon the presence of a programmable crRNA, the Cascade complex targets a genomic locus specifically and stably, which prevents the recruitment or movement of the RNA polymerase (RNAP) and consequently inactivates the expression of the target gene (FIG. 6B). To avoid introducing additional plasmids for crRNA expression, we designed a programmable mini-CRISPR insertion site downstream of the Ptat promoter in the transferable system so that crRNA can be constitutively expressed from the chromosome to execute robust Cascade targeting (FIG. 6A).

Similarly, the rhlI gene in the PAO1 strain was selected to develop this transferable CRISPRi system. We designed five mini-CRISPRs that encode crRNAs targeting different transcriptional regions of the rhlI gene (FIG. 6C). The first crRNA (crRNA-1) targets to the RNAP binding region (Ps-1), crRNA-2 targets to the transcription initiation region (Ps-2), crRNA-3 targets to the 5′-end of the rhlI coding region (Ps-3), crRNA-4 targets to the middle region of the rhlI gene (Ps-4) and crRNA-5 targets to the 3′-end of the rhlI coding region (Ps-5). We assembled these mini-CRISPRs into the transferable CRISPRi plasmid, respectively, and integrated these five plasmids as well as a control plasmid (without mini-CRISPR) into the genome of PAO1. Next, we randomly selected 3 colonies recovered from introduction of each plasmid and quantified their PYO production levels. As expected, PYO production was regulated by the transferable CRISPRi system with the most significant repression of the targeting regions located at the transcription initiation site and the 5′-end of the rhlI coding region (FIG. 6D). There was no repression effect was observed with the crRNA-5 located at 3′-end of the gene. Consistently, transcription levels were significantly decreased in the presence of the crRNA-2 and 3 (FIG. 6F). These results indicated the feasibility of harnessing the transferable CRISPRi system for gene repression and the most effective repression requires the Cascade targeting at the transcription initiation site or its proximal region.

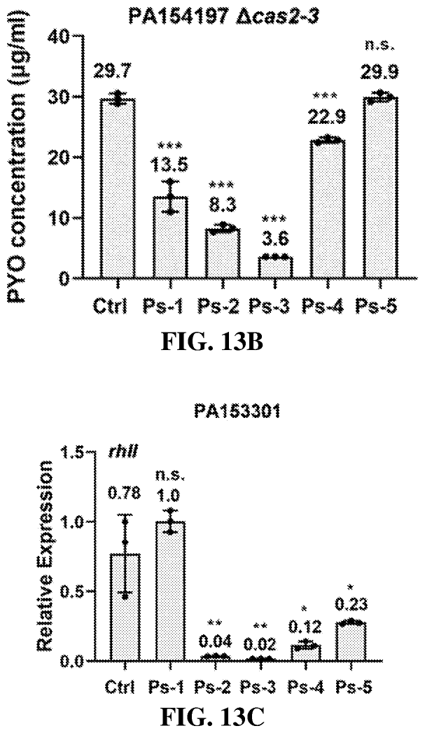

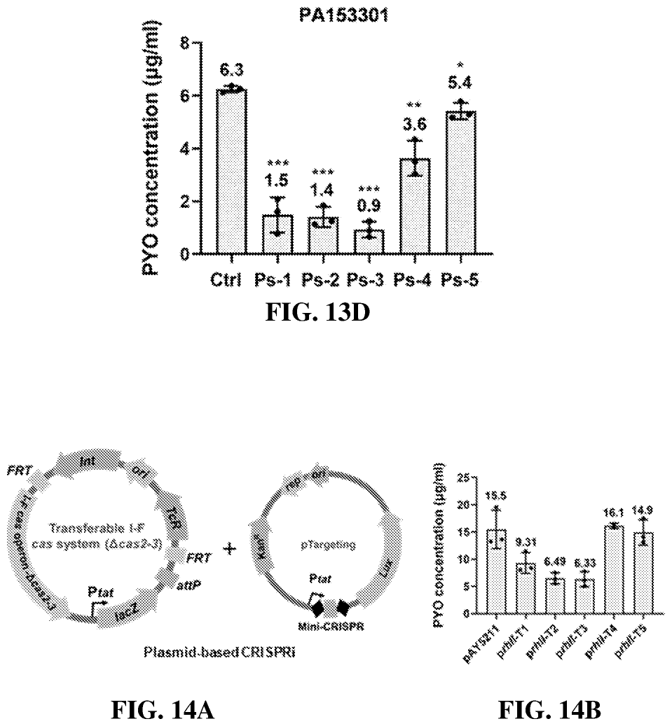

To prove the applicability of the transferable CRISPRi system in other strains, we next examined its capacity in PA27853. An anti-CRISPR gene was detected and first removed from the genome using the counter-selection-based method in this strain, generating a new strain PA27853 ΔacrIF. Same as PAO1, transferable CRISPRi systems that carry the mini-CRISPRs encoding crRNA-2 and 3, respectively, exhibit the most significant reduction of the PYO production and transcription level of rhlI (FIGS. 6E and 6G). This result demonstrated that the transferable CRISPRi system overcomes the limitation of plasmid unavailability and incompatibility, providing a new gene knockdown strategy for functional genomics. Furthermore, the robustness of this repression system was exhibited in other strains such as PA154197 with the deletion of cas2-3 and PA153301 (FIG. 13).

Combining the phage λ-red recombination system, the precise and highly specific genetic manipulations in diverse P. aeruginosa strains with distinct genetic background including strains without native CRISPR-Cas system and strains with native I-F CRISPR-Cas systems but showing different levels of activity was demonstrated. This integration-mediated transferable strategy for Cas expression has several advantages compared with plasmid-encoded Cas proteins.

First, cas machinery can be transferred from E. coli SM10 using RP4 plasmid conjugative machinery efficiently and stably into the exclusive attB site in the genomes of P. aeruginosa strains, which has broader host-range than expression plasmids (Becher & Schweizer, BioTechniques 29, 948-950, 952, doi:10.2144/00295bm04 (2000); Peters, et al., Nature Microbiology 4, 244-250, doi:10.1038/s41564-018-0327-z (2019)) and the whole integrated system can be excised with convenient selection after editing. Second, plasmid-carried cas genes require specific antibiotics to maintain its propagation and Cas expression, which has limited choice for clinical multidrug-resistant isolates and sometimes antibiotic treatment inhibits cell growth or even causes cell lysis during cell proliferation. For the same reasons, the transferable CRISPRi system was established to be free of additional plasmid for crRNA expression, ensuring the robustness and stability of gene repression in diverse hosts without antibiotic treatment. As use of chromosome-encoded Cas subunits in combination with plasmid-encoded crRNA was attempted to repress rhlI expression in PA154197 Acas2-3, cell lysis occurred during cell proliferation in the presence of antibiotic. Although CRISPRi-based reduction of PYO production we observed (FIG. 14), cell lysis impeded the next-step of transcriptional analysis.

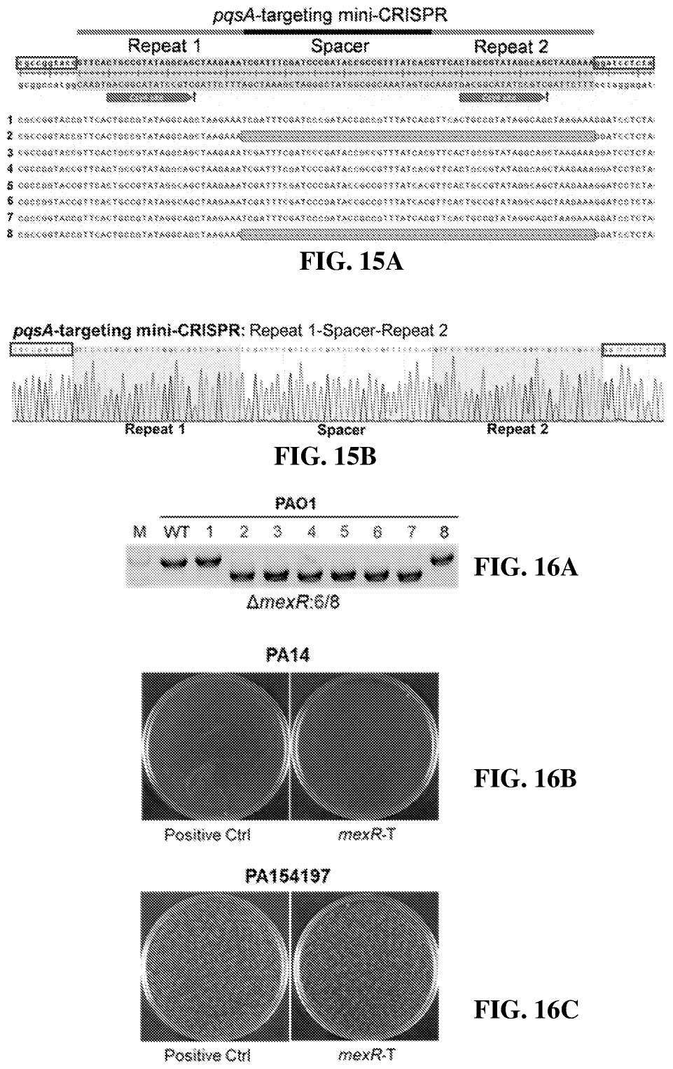

Although self-targeting assay showed significantly decreased conjugation efficiency by introducing the targeting plasmid into the transferable I-F cas system integrated strains compared with the introduction of the control plasmid pAY5211, colonies still occurred by escaping self-targeting. For example, in the PAO1 strain, around 107 colonies were recovered from the transformation of pAY5211 and 27 colonies were recovered in average from pRhlI-Targeting. These colonies escaped from targeting constitute the major false positive clones during editing and consequently reduce the editing efficiency. Occurrence of false positive clones is mainly attributed to the mutations in the self-targeting mini-CRISPR or cas genes. As the genomes of three representative false positive clones in rhlI deletion were sequenced, mutations in the cas genes in all the three clones were identified. Although mutations in the rhlI-targeting mini-CRISPRs were not found, mutations in the pqsA-targeting mini-CRISPR were observed when they were recovered and sequenced from 8 false positive clones in the case of pqsA deletion. Two of them showed the loss of the spacer and one repeat sequence (FIG. 15). Understanding and preventing these spontaneous mutations will help to increase the efficiency in both targeting and editing.

Successful gene deletions were achieved using the transferable I-F cas system, but the efficiency varies in diverse P. aeruginosa hosts. For example, in the PAO1 strain and the PA130788 strain with the deletion of anti-CRISPR element, gene deletion can be achieved at the rate higher than 80%. However, low efficiency ranging from 20% to 40% were observed in other strains such as PA150577, PA151671 and PA132533. The transferred cas genes in these strains showed comparable expression levels (FIG. 9A), indicating that additional factors may influence the editing efficiency such as the mutation frequency as discussed above. In addition, induction of exogenous recombination systems requires optimization in different strain. Moreover, the result did not show that multiple spacers could promote editing efficiency as comparable editing efficiency was obtained using the editing plasmid containing single or double effective spacers. Nonetheless, multiple spacers could still increase the targeting potentials to avoid non-effective targeting at some special locus within the region to be deleted.

The transferred CRISPR-Cas systems are not active in all the strains. The systems were only active in 10 out of 30 clinical strains. Given that a high percentage of sequenced P. aeruginosa genomes (30%) contains anti-CRISPR genes, anti-CRISPR elements are speculated as the major obstacle in CRISPR-Cas exploitations in prokaryotes. After searching anti-CRISPR genes in two stains, PA130788 and PA27853, whose whole genome sequences are available and their transferred CRISPR-Cas systems are inactive, both genomes were found to contain an anti-CRISPR gene. Activities of the CRISPR-Cas systems in these two strains were reactivated by deleting their endogenous anti-CRISPR genes for efficient gene deletion and gene repression, respectively. Based on the obtained results, removal of the anti-CRISPR element by other methods such as counterselection-based method exhibited the most effective way to overcome the inactivation of CRISPR-Cas in specific strains. Even though removal of the anti-CRISPR element is relatively laborious and time-consuming using these methods, one-step transferable CRISPR-Cas-based genome editing or gene repression can be achieved once the anti-CRISPR element is removed. However, this strategy is not applicable in the strains without applicable genetic tools. Over-expression of anti-CRISPR repressors seems the most promising strategy to reactive CRISPR-Cas system by introducing a plasmid to express the associated repressor gene aca. However, when repression of the anti-CRISPR gene by over-expressing its associated repressor Aca in PA130788 was attempted, it was still not possible to reactivate the CRISPR-Cas system efficiently for genetic manipulation even with 28-fold upregulated expression of aca. This means that Aca-based repression is not robust, and the repression efficiency may vary in different strains possibly owing to the distinct genetic background.

Cas9-based two-step approaches were developed to achieve efficient genome editing in P. aeruginosa strains PAO1 and PAK. However, they failed to deliver desired gene deletion in other well-characterized strains such as PA14 and PA154197 by implementing the editing plasmid that was used to efficiently delete the mexR gene in PAO1. Difficulty to transform the editing plasmid into PA14 and dysfunctionality of the Cas9 system in PA154197 resulted in the failure of gene deletion in two strains, respectively (FIG. 16). Given that the simplicity of Cas9 in DNA interference, use of the strategy of chromosomal cas9-integration to stabilize the cas9 expression was still expected. However, the chromosome-encoded Cas9 failed to generate effective self-targeting with unknown reasons. These results highlight the advantage of the type I CRISPR-Cas system that was employed for genetic manipulations in bacterial cells.

### REFERENCES

1 Marraffini, L. A. CRISPR-Cas immunity in prokaryotes. Nature 526, 55-61, doi:10.1038/nature15386 (2015).
2 Makarova, K. S. et al. Evolutionary classification of CRISPR-Cas systems: a burst of class 2 and derived variants. Nature Reviews Microbiology 18, 67-83, doi:10.1038/s41579-019-0299-x (2020).
3 Koonin, E. V. & Makarova, K. S. Origins and evolution of CRISPR-Cas systems. Philosophical Transactions of the Royal Society B—Biological Sciences 374, doi:10.1098/rstb.2018.0087 (2019).
4 Adli, M. The CRISPR tool kit for genome editing and beyond. Nature Communications 9, doi:10.1038/s41467-018-04252-2 (2018).
5 Knott, G. J. & Doudna, J. A. CRISPR-Cas guides the future of genetic engineering. Science 361, 866-869, doi:10.1126/science.aat5011 (2018).
6 Zhang, F. Development of CRISPR-Cas systems for genome editing and beyond. Quarterly Reviews of Biophysics 52, doi:10.1017/s0033583519000052 (2019).
7 Burstein, D. et al. New CRISPR-Cas systems from uncultivated microbes. Nature 542, 237-241, doi:10.1038/nature21059 (2017).
8 Koonin, E. V., Makarova, K. S. & Zhang, F. Diversity, classification and evolution of CRISPR-Cas systems. Current Opinion in Microbiology 37, 67-78, doi:10.1016/j.mib.2017.05.008 (2017).
9 Pickar-Oliver, A. et al. Targeted transcriptional modulation with type I CRISPR-Cas systems in human cells. Nature Biotechnology 37, 1493-1501, doi:10.1038/s41587-019-0235-7 (2019).
10 Chen, Y. et al. Repurposing type I-F CRISPR-Cas system as a transcriptional activation tool in human cells. Nature Communications 11, 3136, doi:10.1038/s41467-020-16880-8 (2020).
11 Dolan, A. E. et al. Introducing a Spectrum of Long-Range Genomic Deletions in Human Embryonic Stem Cells Using Type I CRISPR-Cas. Molecular Cell 74, 936-+, doi:10.1016/j.molcel.2019.03.014 (2019).
12 Cameron, P. et al. Harnessing type I CRISPR-Cas systems for genome engineering in human cells. Nature Biotechnology 37, 1471-+, doi:10.1038/s41587-019-0310-0 (2019).
13 Xu, Z., Li, Y., Li, M., Xiang, H. & Yan, A. Harnessing the type I CRISPR-Cas systems for genome editing in prokaryotes. Environmental Microbiology n/a, doi:10.1111/1462-2920.15116 (2020).
14 Li, Y. J. et al. Harnessing Type I and Type III CRISPR-Cas systems for genome editing. Nucleic Acids Research 44, doi:10.1093/nar/gkv1044 (2016).
15 Pyne, M. E., Bruder, M. R., Moo-Young, M., Chung, D. A. & Chou, C. P. Harnessing heterologous and endogenous CRISPR-Cas machineries for efficient markerless genome editing in Clostridium. Scientific Reports 6, doi:10.1038/srep25666 (2016).
16 Hidalgo-Cantabrana, C., Goh, Y. J., Pan, M. C., Sanozky-Dawes, R. & Barrangou, R. Genome editing using the endogenous type I CRISPR-Cas system in Lactobacillus crispatus. Proceedings of the National Academy of Sciences of the United States of America 116, 15774-15783, doi:10.1073/pnas.1905421116 (2019).
17 Xu, Z. L. et al. Native CRISPR-Cas-Mediated Genome Editing Enables Dissecting and Sensitizing Clinical Multidrug-Resistant P. aeruginosa. Cell Reports 29, 1707-+, doi:10.1016/j.celrep.2019.10.006 (2019).
18 Selle, K. & Barrangou, R. Harnessing CRISPR-Cas systems for bacterial genome editing. Trends in Microbiology 23, 225-232, doi:10.1016/j.tim.2015.01.008 (2015).
19 Oliver, A., Mulet, X., López-Causapé, C. & Juan, C. The increasing threat of Pseudomonas aeruginosa high-risk clones. Drug resistance updates: reviews and commentaries in antimicrobial and anticancer chemotherapy 21-22, 41-59, doi:10.1016/j.drup.2015.08.002 (2015).
20 Silby, M. W., Winstanley, C., Godfrey, S. A., Levy, S. B. & Jackson, R. W. Pseudomonas genomes: diverse and adaptable. FEMS microbiology reviews 35, 652-680, doi:10.1111/j.1574-6976.2011.00269.x (2011).
21 Hidalgo-Cantabrana, C. & Barrangou, R. Characterization and applications of Type I CRISPR-Cas systems. Biochemical Society Transactions 48, 15-23, doi:10.1042/bst20190119 (2020).
22 Richter, C. et al. Priming in the Type I-F CRISPR-Cas system triggers strand-independent spacer acquisition, bi-directionally from the primed protospacer. Nucleic Acids Res 42, 8516-8526, doi:10.1093/nar/gku527 (2014).
23 Richter, C., Gristwood, T., Clulow, J. S. & Fineran, P. C. In vivo protein interactions and complex formation in the Pectobacterium atrosepticum subtype I-F CRISPR/Cas System. PloS one 7, e49549, doi:10.1371/journal.pone.0049549 (2012).
24 Xu, Z., Li, Y. & Yan, A. Repurposing the Native Type I-F CRISPR-Cas System in Pseudomonas aeruginosa for Genome Editing. STAR Protocols 1, 100039, doi:https://doi.org/10.1016/j.xpro.2020.100039 (2020).
25 Duan, K. & Surette, M. G. Environmental regulation of Pseudomonas aeruginosa PAO1 las and rhl quorum-sensing systems. Journal of Bacteriology 189, 4827-4836, doi:10.1128/jb.00043-07 (2007).
26 Grohmann, E., Muth, G. & Espinosa, M. Conjugative plasmid transfer in gram-positive bacteria. Microbiology and Molecular Biology Reviews 67, 277-+, doi:10.1128/mmbr.67.2.277-301.2003 (2003).
27 Chen, W. et al. CRISPR/Cas9-based Genome Editing in Pseudomonas aeruginosa and Cytidine Deaminase-Mediated Base Editing in Pseudomonas Species. iScience 6, 222-231, doi:10.1016/j.isci.2018.07.024 (2018).
28 Peng, X., Mayo-Munoz, D., Bhoobalan-Chitty, Y. & Martinez-Alvarez, L. Anti-CRISPR Proteins in Archaea. Trends in Microbiology, doi:10.1016/j.tim.2020.05.007.
29 Marino, N. D., Pinilla-Redondo, R., Csorgo, B. & Bondy-Denomy, J. Anti-CRISPR protein applications: natural brakes for CRISPR-Cas technologies. Nature Methods 17, 471-479, doi:10.1038/s41592-020-0771-6 (2020).
30 van Belkum, A. et al. Phylogenetic Distribution of CRISPR-Cas Systems in Antibiotic-Resistant Pseudomonas aeruginosa. Mbio 6, doi:10.1128/mBio.01796-15 (2015).
31 Yi, H. et al. AcrFinder: genome mining anti-CRISPR operons in prokaryotes and their viruses. Nucleic Acids Research 48, W358-W365, doi:10.1093/nar/gkaa351% J Nucleic Acids Research (2020).
32 Choi, K.-H. & Schweizer, H. P. An improved method for rapid generation of unmarked Pseudomonas aeruginosa deletion mutants. BMC Microbiol 5, 30-30, doi:10.1186/1471-2180-5-30 (2005).
33 Stanley, S. Y. et al. Anti-CRISPR-Associated Proteins Are Crucial Repressors of Anti-CRISPR Transcription. Cell 178, 1452-1464.e1413, doi:https://doi.org/10.1016/j.cell.2019.07.046 (2019).
34 Csörgő, B. et al. A minimal CRISPR-Cas3 system for genome engineering. bioRxiv, 860999, doi:10.1101/860999 (2019).
35 Bikard, D. et al. Programmable repression and activation of bacterial gene expression using an engineered CRISPR-Cas system. Nucleic acids research 41, 7429-7437, doi:10.1093/nar/gkt520 (2013).
36 Becher, A. & Schweizer, H. P. Integration-proficient Pseudomonas aeruginosa vectors for isolation of single-copy chromosomal lacZ and lux gene fusions. BioTechniques 29, 948-950, 952, doi:10.2144/00295bm04 (2000).
37 Peters, J. M. et al. Enabling genetic analysis of diverse bacteria with Mobile-CRISPRi. Nature Microbiology 4, 244-250, doi:10.1038/s41564-018-0327-z (2019).
38 Xu, Z. L. et al. Zinc excess increases cellular demand for iron and decreases tolerance to copper in Escherichia coli. Journal of Biological Chemistry 294, 16978-16991, doi:10.1074/jbc.RA119.010023 (2019).
39 Bolger, A. M., Lohse, M. & Usadel, B. Trimmomatic: a flexible trimmer for Illumina sequence data. Bioinformatics 30, 2114-2120, doi:10.1093/bioinformatics/btu170 (2014).
40 Bankevich, A. et al. SPAdes: A New Genome Assembly Algorithm and Its Applications to Single-Cell Sequencing. J Comput Biol 19, 455-477, doi:10.1089/cmb.2012.0021 (2012).
41 Page, A. J. et al. Robust high-throughput prokaryote de novo assembly and improvement pipeline for Illumina data. doi:10.17863/CAM.43554 (2016).
42 Hunt, M. et al. Circlator: automated circularization of genome assemblies using long sequencing reads. Genome Biol 16, 294, doi:10.1186/s13059-015-0849-0 (2015).
43 Seemann, T. Prokka: rapid prokaryotic genome annotation. Bioinformatics 30, 2068-2069, doi:10.1093/bioinformatics/btu153 (2014).
44 Klompe, S. E., Vo, P. L. H., Halpin-Healy, T. S. & Sternberg, S. H. Transposon-encoded CRISPR-Cas systems direct RNA-guided DNA integration. Nature 571, 219-225, doi:10.1038/s41586-019-1323-z (2019).
45 Li, H. Aligning sequence reads, clone sequences and assembly contigs with BWA-MEM. (2013).
46 Li, H. et al. The Sequence Alignment/Map format and SAMtools. Bioinformatics 25, 2078-2079, doi:10.1093/bioinformatics/btp352 (2009).
47 Quinlan, A. R. & Hall, I. M. BEDTools: a flexible suite of utilities for comparing genomic features. Bioinformatics 26, 841-842, doi:10.1093/bioinformatics/btq033 (2010).
48 Cao, H. L. et al. Uncoupled Quorum Sensing Modulates the Interplay of Virulence and Resistance in a Multidrug-Resistant Clinical Pseudomonas aeruginosa Isolate Belonging to the MLST550 Clonal Complex. Antimicrobial Agents and Chemotherapy 63, doi:10.1128/aac.01944-18 (2019).

1. A system for changing, adding, and/or deleting one or more genes in a microbial cell, comprising
(a) a nucleic acid type I-F cas system vector, comprising
(i) a type I-F cas operon, comprising the cas genes cas1, cas2-3, cas8f, cas5, cas7 and cas6, and a nucleic acid sequence configured to promote transcription of the cas genes in the microbial cell;
(ii) one or more genes encoding an integrase enzyme;
(iii) a nucleic acid sequence recognized as an integration site configured to recognize and attach the vector to a target attachment site within the microbial genome;
(iv) two nucleic acid sequences configured to be Flp recombinase target sites; and
(v) one or more reporter genes and a nucleic acid sequence configured to promote transcription of the reporter gene(s) upon integration into the microbial cell genome;
wherein the vector is configured for integration into the genome of the microbial cell via attachment at the target attachment site; and
(b) a nucleic acid editing vector comprising one or more CRISPR RNA (crRNA) nucleic acids and repair donors, configured to change, add, and/or delete one or more target genomic sites in the microbial cell through the type I-F CRISPR-Cas system.

2. The system of claim 1, further comprising
(c) a nucleic acid targeting vector, comprising one or more CRISPR RNA (crRNA) nucleic acids configured to disrupt one or more target genomic sites in the microbial cell required for transcription and/or expression of the one or more reporter genes within the type I-F cas system vector.

3. The system of claim 1, wherein the one or more reporter genes within the nucleic acid type I-F cas system vector is a lacZ reporter gene.

4. The system of claim 2, wherein the targeting vector comprises crRNA nucleic acids configured to disrupt one or more genes associated with expression of the Acyl-homoserine-lactone synthase enzyme.

5. The system of claim 1, wherein the type I-F cas operon is the type I-F cas operon from P. aeruginosa strain PA154197.

6. The system of claim 1, wherein the target attachment site within the microbial genome is the attB site of Pseudomonas aeruginosa.

7. The system of claim 1, wherein the nucleic acid I-F cas system vector further comprises:
(vi) a functional phage λ-red recombination system, comprising genes encoding λ-Red proteins Exo, Gam, and Beta, and an arabinose-inducible promoter.

8. The system of claim 1, further comprising
(c) a nucleic acid CRISPR-Cas removal vector, comprising one or more CRISPR RNA (crRNA) nucleic acids configured to delete an integrated type I-F cas system from the microbial cell.

9. A system for repressing a gene of interest in a microbial cell, comprising a nucleic acid type I-F CRISPRi system vector comprising
(i) a type I-F cas operon, comprising the cas genes cas1, cas8f, cas5, cas7, and cas6, and a nucleic acid sequence configured to promote transcription of the cas genes in the microbial cell, wherein the type I-F cas operon lacks a functional copy of cas2-3 gene;
(ii) one or more genes encoding an integrase enzyme;
(iii) a nucleic acid sequence encoding an integration site configured to recognize and attach the plasmid to a target attachment site within the microbial genome;
(iv) two nucleic acid sequences configured to be Flp recombinase target sites;
(v) one or more reporter genes and a nucleic acid sequence configured to promote transcription of the reporter gene(s) upon integration into the microbial cell genome; and
(vi) one or more CRISPR RNA (crRNA) nucleic acids, configured to target one or more sites of the gene of interest in the genome of the microbial cell,
wherein the vector is configured for integration into the genome of the microbial cell via attachment at the target attachment site.

10. The system of claim 9, wherein the functional copy of cas2-3 gene is absent from the type I-F cas operon.

11. The system of claim 9, wherein the one or more reporter genes within the nucleic acid type I-F CRISPRi system vector is a lac Z reporter gene.

12. The system of claim 9, wherein the type I-F cas operon is based on the I-F cas operon from P. aeruginosa strain PA154197.

13. The system of claim 9, wherein the target attachment site within the microbial genome is the attB site of Pseudomonas aeruginosa.

14. The system of claim 9, wherein the one or more CRISPR RNA nucleic acids are configured to target one or more transcriptional sites of the gene of interest.

15. The system of claim 14, wherein the one or more transcriptional sites are selected from the group consisting of the RNA polymerase binding region, the transcription initiation region, the 5′-end of the coding region, the middle region of the gene, the 3′-end of the coding region, or a combination thereof, of the gene of interest in the recipient cell.

16. A microbial cell comprising the recombinant nucleic acid type I-F CRISPR-Cas system of claim 1, the system comprising.

17. The microbial cell of claim 16, wherein the cell is a Pseudomonas spp. Bacterium, wherein the cell is a P. aeruginosa bacterium, or wherein the cell is a P. aeruginosa bacterium that is a strain selected from strain PAO1, strain PA14, strain PA27853, strain PA150577, strain PA154197, strain PA151671, strain PA130788, and strain PA132533.

18-19. (canceled)

20. The microbial cell of claim 16, wherein the cell does not contain an endogenous CRISPR-Cas system.

21. The microbial cell of claim 16, wherein the cell does not comprise functional anti-CRISPR genes, or other components that inactivate the type I-F CRISPR-Cas system.

22. The microbial cell of claim 16, wherein the cell is a P. aeruginosa strain bacterium or a P. aeruginosa strain PA130788, wherein endogenous anti-CRISPR elements have been disrupted.

23. (canceled)

24. A method for changing, adding, and/or deleting one or more genes in a microbial cell using the system of claim 1, comprising the steps of
(a) contacting the bacterial cell with the nucleic acid type I-F cas system vector,
wherein the contacting occurs under conditions suitable for integration of the type I-F CRISPR-Cas system into the microbial cell genome to form a recipient cell; and
(b) contacting the recipient cell with the nucleic acid editing vector.

25. The method of claim 24, wherein the nucleic acid I-F cas system vector further comprises
(vi) a functional phage λ-red recombination system, comprising genes encoding λ-Red proteins Exo, Gam, and Beta, and an arabinose-inducible promoter.

26. The method of claim 24, wherein the type I-F CRISPR-Cas system induces from about 1%, 2%, 3%, 4%, 5%, 6%, 7%, 8%, 9%, 10%, 20%, 30%, 40%, 50%, 60%, 70%, 80%, 90%, up to 100% reduction in the expression, translation, or activity of a target gene in the recipient cell.

27. The method of claim 24, further comprising the step of
(c) assessing the activity of the integrated Cas system (a) by contacting the recipient cell with a nucleic acid targeting vector, comprising one or more CRISPR RNA (crRNA) nucleic acids configured to disrupt one or more target genomic sites in the microbial cell required for transcription and/or expression of the one or more reporter genes within the type I-F CRISPR-Cas system vector, wherein efficiency is assessed by efficacy of the CRISPR-mediated genome interference.

28. The method of claim 24, wherein the one or more reporter genes within the nucleic acid type I-F cas system vector is a lac Z reporter gene.

29. The method of claim 24, further comprising the step of
(d) removing the CRISPR-Cas system from the recipient cell by contacting the recipient cell with a nucleic acid CRISPR-Cas removal vector, wherein the vector comprises a lacZ-targeting mini-CRISPR and a donor sequence downstream of homologous arms of the attB insertion site.

30. A method for repressing a gene of interest in a microbial cell using the system of claim 1, comprising the step of contacting the bacterial cell with the nucleic acid type I-F CRISPRi system vector,
wherein the contacting occurs under conditions suitable for integration of the type I-F CRISPRi system into the microbial cell genome to form a recipient cell.

31. The method of claim 30, wherein the type I-F CRISPRi system induces from about 1%, 2%, 3%, 4%, 5%, 6%, 7%, 8%, 9%, 10%, 20%, 30%, 40%, 50%, 60%, 70%, 80%, 90%, up to 100% reduction in the transcription of the gene of interest in the recipient cell.
